(19) United States
(12) Patent Application Publication
(10) Pub. No.: US 2014/0342946 A1
(43) Pub. Date: Nov. 20, 2014

(54) DIAGNOSTIC TESTS FOR PREDICTING PROGNOSIS, RECURRENCE, RESISTANCE OR SENSITIVITY TO THERAPY AND METASTATIC STATUS IN CANCER

(71) Applicants: Moni Abraham KURIAKOSE, (US); Amritha SURESH, (US)

(72) Inventors: Moni Abraham Kuriakose, Ernakulam (IN); Amritha Suresh, Bangalore (IN)

(21) Appl. No.: 14/368,801

(22) PCT Filed: Dec. 31, 2012
(86) PCT No.: PCT/IB2012/057844

§ 371 Date: Jun. 26, 2014

Related U.S. Application Data
(60) Provisional application No. 61/631,291, filed on Dec. 31, 2011.

(51) Int. Cl. C12Q 1/68

(52) U.S. Cl. CPC C12Q 1/68
USPC 506/17

(57) ABSTRACT

The present invention describes a method utilizing a set of genes or gene products whose altered expression in cancer tissue, particularly head and neck cancer and other carcinomas, or its adjacent normal tissues predicts (a) probability of recurrence in time after treatment (b) sensitivity or resistance to therapies or (c) probability of metastasis at the time of initial discovery of the tumor. Furthermore, the invention describes methods of determining the molecular signature in tumor tissues, tissues adjacent to the tumor, or in saliva by using DNA microarray techniques, quantitative real-time PCR, immunohistochemistry or other methods that are used for determining gene or gene product expression levels.

### CROSS REFERENCE TO RELATED APPLICATION

The present application is a U.S. national stage application (under 35 USC §§371) of PCT international application PCT/IB2012/057844 having an international filing date 31 Dec. 2012, which claims priority from U.S. provisional application No. 61/631,291 filed with USPTO on 31 Dec. 2011.

### TECHNICAL FIELD OF INVENTION

The present invention relates to a process for personalization of cancer treatment involving the use of specific genes and/or their proteins in diagnostic tests for predicting prognosis, recurrence, resistance or sensitivity to therapy and metastasis status in cancer.

### BACKGROUND OF THE INVENTION

Cancer and its progression in an individual is guided by the expression and/or altered status of many genes and gene products (molecular markers). Correlation of the changes in these molecular markers can help to predict if a particular patients cancer would (a) recur in time after treatment or (b) be sensitive or resistant to therapies or (c) have metastasized at the time of initial discovery of the tumor, consequently leading to improved ability to manage cancer.

More recently, molecular signatures have been described as a more robust tool for determining prognosis or metastatic status. Companies such as Genomic Health Inc. and Agendia have introduced similar diagnostic tools (Oncotype DX and Mammaprint respectively) in the market for Breast cancer and colorectal cancer (U.S. Pat. No. 7,930,104, WO2009/114836, WO2009/002175A1). However, analogues molecular signature for head and neck cancers are limited. U.S. Pat. No. 7,588,895 looks at an eight gene signature in oral squamous cell carcinoma predicting metastasis and extra capsular spread, while patent no. WO2007/015935A2 uses a twelve gene signature for predicting therapeutic success, recurrence free and overall survival. The set of genes in the present invention is exclusive of the genes in above mentioned inventions.

Development of efficient assays determining the probability that a tumor is likely to recur in a short time or a tumor would be resistant to cytotoxic therapy or radiation, would help the physician to seek other choices for therapy rather than subject the patient to treatments that would have no benefit. Identification of a set of markers that would predict cancers resistant to treatment modalities and hence susceptible for recurrent behavior or that can predict whether a tumor has metastasized or not will have significant clinical benefit. Further, their detection in accessible body fluids such as in saliva would be a significant advantage.

### DETAILED DESCRIPTION OF THE INVENTION

In order to more clearly and concisely describe and point out the subject matter of the claimed invention, the following definitions are provided for specific terms which are used in the following written description.

“Molecular signature” refers to the expression of two or more genes described in Tables I-V, or more specifically Table X, in a tumor tissue or in tumor cells derived from tongue or other head-and-neck cancers; the said gene expression level being determined by one or more techniques that is commonly employed for measuring gene expression levels in tissues or cells which includes microarrays and real-time quantitative polymerase chain reaction. Levels of gene expression could also be determined by measuring the level of proteins encoded by the said genes using immunohistochemistry, enzyme-linked-immunosorbent assay or other methods like proteomic techniques for mapping expression of multiple proteins.

“Molecularly-targeted therapies” shall mean a treatment modality against cancer cells targeting specific molecules involved in tumorigenesis and tumor growth.

“Immune modulation therapy” shall mean the use of modulators that inhibit/stimulate the immune system to elicit anti-tumor effects. In the present invention tongue cancer is used as an example of head and neck cancer and other carcinomas to describe a method utilizing a set of genes or gene products whose altered expression, in head and neck tumor in general including tongue cancer, predicts (a) probability of recurrence in time after treatment (b) sensitivity or resistance to therapies or (c) probability of metastasis at the time of initial discovery of the tumor,

The novel molecular signature comprises of a combination of genes selected from the list of genes given in Tables I-V or a narrower set of more differentially expressed genes from a preferred list of genes drawn from Tables I-V and listed in Table X.

In accordance with preferred embodiments, the molecular signature is identified in pre-treatment and post-treatment head and neck cancer and is used to determine the probability of recurrence of cancer after surgery and anti-cancer therapy. Absence of the molecular signature in the primary tumor sample would imply a far less probability of recurrence; hence one could avoid further therapy after surgery, thus decreasing the cost of treatment as well as the morbidity associated with chemotherapy. Presence of the molecular signature in the tumor at the time of surgery would reveal a higher probability of recurrence and therefore would aid in determining if adjuvant chemotherapy is warranted or not.

In another embodiment of the invention, the molecular signature is used to identify sensitivity or resistance to anti-cancer agents, in particular chemotherapy agents, but not limited to the same, and would include radiation therapy or new generation molecularly-targeted drugs or immune-modulating drugs or cell therapy like dendrite cell therapy.

The present invention also identifies a molecular signature, listed in Table V, which is differentially expressed in the adjacent histologically normal mucosa of the recurrent and non-recurrent patients. This molecular signature describes groups of cells in the adjacent mucosa of the recurrent patients that show the over expression of stem cell markers and transcription factors. The presence of these cells, as identified by the molecular signature, in the adjacent mucosa could also be predictive of recurrence in patients with head and neck cancer.

In yet another embodiment of the invention, the molecular signature is used to determine the probability that a tumor would has metastasized to a secondary location at the time of diagnosis of the disease, which will allow one to determine if surgery alone is sufficient or adjuvant chemotherapy or other anti-cancer drugs or therapies are required. The molecular signature in Table I-V, and more specifically Table X describes characteristics of the tumor that can be also used to predict if the cancer has metastasized to a secondary location by virtue of (a) the fact that the molecular signature identifies aggressive cells in the tumor that by definition has a higher invasive potential (b) the immune repressive genes that are over-expressed would allow the tumor to escape its primary site and metastasize.

The same is indicated through the pathway enrichments seen using Ingenuity pathway analysis between the groups; Group I (Pre-treatment, non-recurrent and from Group III (post-treatment, recurrent); with p<0.05 and Fishers exact test applied as a test of significance. The top 10 canonical pathways identified in the recurrent and the non-recurrent groups after core comparison analysis are represented in FIGS. 3A and 3B. The most significant pathways include Glioma invasiveness signaling, bladder cancer signaling, LXR/RXR activation and colorectal cancer metastasis signaling in the recurrent group. In comparison, the non-recurrent set primarily showed Interferon signaling; Cytotoxic T-lymphocyte mediated apoptosis of target cells, protein ubiquitination and Myc mediated apoptosis as significant pathways. Genes differentiating between recurrent and non-recurrent tumors, listed in tables III-IV, therefore are enriched in candidates that can predict invasiveness and metastasis.

The individual genes and gene-products of the molecular signature discussed in this invention, and listed in Tables I to V, have been identified as serving key functions in disease recurrence and resistance or sensitivity to chemotherapy as well as metastasis in a large array of cancers like lung, pancreas, colorectal, hepatocellular carcinoma, breast, ovarian, melanoma, glioma, neuroblastoma, endometrial, prostate, lymphoma and a variety of other cancers. In other words the invention described herein is broadly applicable to most cancers and all carcinomas and not just tongue or head and neck cancer.

In another embodiment of the invention, the tumor tissue that is used for analysis include tissue biopsies—either frozen, fixed in RNA stabilizing solutions or in paraffin-embedded-formalin fixed tissues (FFPE), or saliva which is used as the source RNA or protein for determination of the molecular signature

In another embodiment of the invention, the assays used for determining the molecular signature includes microarray, quantitative real-time PCR, immunohisotochemisitry, enzyme-linked immunosorbent assay, proteomic analysis or other standard methods of measuring gene expression of multiple directly or through proteins encoded by the genes.

### BRIEF DESCRIPTION OF TABLES AND DRAWINGS

In order that the invention be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present invention.
Table I: Differentially expressed genes in the oral tongue tumors (p<0.05)
Table II: Differentially expressed genes in the non-recurrent oral tongue tumors (p<0.05)
Table III: Differentially expressed genes in the recurrent oral tongue tumors (p<0.05)
Table IV: Differentially expressed genes between Non-recurrent Tumor and Recurrent Tumor (p<0.05)
Table V: Differentially expressed genes in the adjacent mucosa (Non Recurrent versus Recurrent) (p<0.05)
Table VI: Clinical Characteristics of patients
Table VII: List of top 10 significant genes in Non-Recurrent/recurrent tongue cancer
Table VIII: List of significant genes in Recurrent tongue cancer
Table IX: Receiver Operating Curve and Regression analysis of the markers
Table X: Consolidated List of genes with high differential expression

FIG. 1 Hierarchical and K-means clustering of differentially expressed genes in recurrent tongue cancer Clustering analysis was done using MeV (TIGR) after application of Wilcoxon Mann Whitney test using the Euclidean distance measurement. The clustering analysis revealed classifiers for recurrent tumors (A) and all tumors (B). K-means clustering (K=10; Euclidean distance) was also carried out with the distinct clusters of immune response genes up regulated in non recurrent tumors (C) and HBA/HBB clusters down regulated in recurrent tumors (D).

FIG. 2 Differential expression in the adjacent mucosal tissue Hierarchical clustering between adjacent mucosal tissue revealed extensive differences in expression profiling (A). K-means clustering showed the up regulation of a sub-set of genes including stem cell genes such as ATR, ARHGAP5 (B) and down regulation HBB/HBA1 cluster in the recurrent patients (C). Statistical analysis (ANOVA) also revealed a sub set of genes overlapping between the adjacent mucosal tissue and tumor samples of the recurrent patients (D).

FIG. 3 Significant pathways between Non-recurrent and recurrent tongue cancer Pathway analysis was carried out by Ingenuity Pathway Analysis (IPA) and the top 10 significant pathways are represented in the figure. The pathways are sorted according to significance in recurrent sub set (A) and non-recurrent samples (B).

FIGS. 4 A and 4 B Interaction networks identified by Ingenuity Pathway Analysis Interaction network of genes that are differentially expressed between Non-recurrent and recurrent tumors (A & B). The symbols in the figure denote the following A: Activation, E: Expression, PP: protein-Protein Interaction, I: Inhibition, L: Proteolysis; P: Phosphorylation, T: Transcription, PD: Protein-DNA interaction. Note the group of genes, the expression of which is dependent upon XBP1 and E2F. The binding partners HBB and HBA1 are both higher in expression in non-recurrent tumors.

FIG. 5 Validation in tissues and saliva samples. The expression profile of a select subset of markers was validated in tongue cancer specimens (A). A distinct difference in expression profile of 4 genes (COL5A1, IGLA, HBBand CTSC) was observed in the primary tissue of patients that were non-recurrent (Group I) and recurrent (Group II). The pattern of expression obtained in the patients of the latter group was similar to that obtained in the recurrent tissue of patients (Group III). ROC analysis revealed these markers as most significant according to the AUC (B). The profile of 6 genes in saliva samples from normal (N) and tumor (T) samples is shown (C). The normal samples primarily show the expression of IL1B while at least one of the carcinogenesis related genes are expressed in the patients. ROC analysis of the combination of markers (ABCG1, IL8, COL5A1, FN1 and MMP1) shows sensitivity of 0.65 and specificity of 0.87 (D).

FIG. 6 Immunohistochemical analysis of candidate markers IHC was carried out on tongue cancer samples (A) with antibodies to HBB (a, b, c, d) and COL5A1 (e, f, g, h). The expression was analyzed in normal controls (a, e), in non-recurrent tumor samples (b & f) and in recurrent samples (c & g). d & h represent negative controls. The non-recurrent tumor sample showed a high expression of HBB as observed in the normal control; while an over expression of COL5A1 was observed in the recurrent tumor sample. The magnifications (100 or 200 times the original magnification) are mentioned on each panel. ROC analysis showed HBB as a better candidate marker as compared to COL5A1 (B & C).

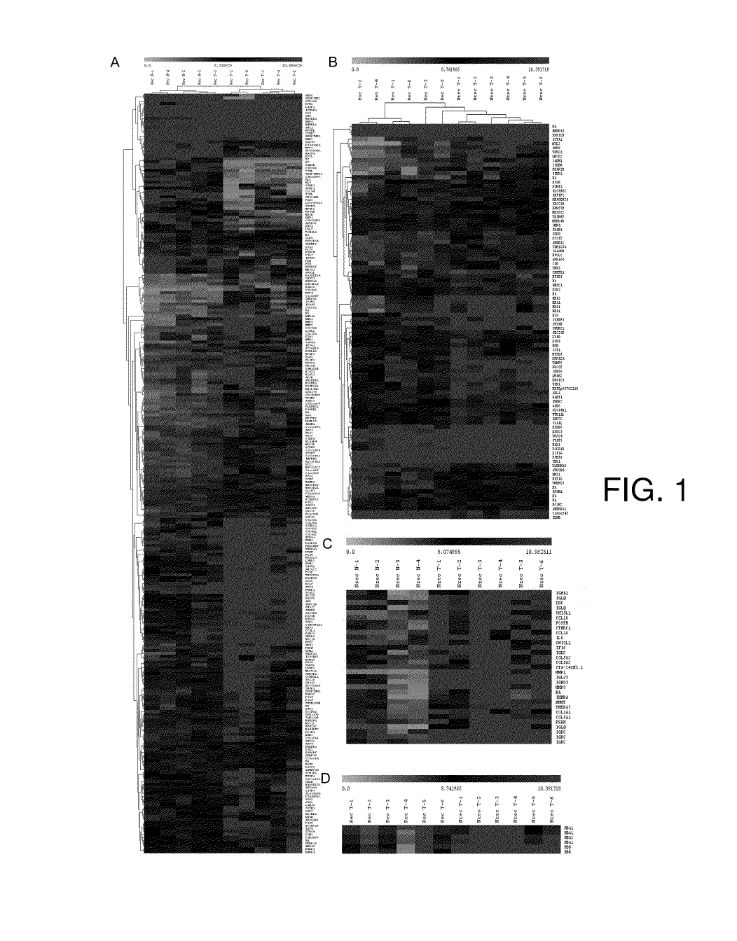

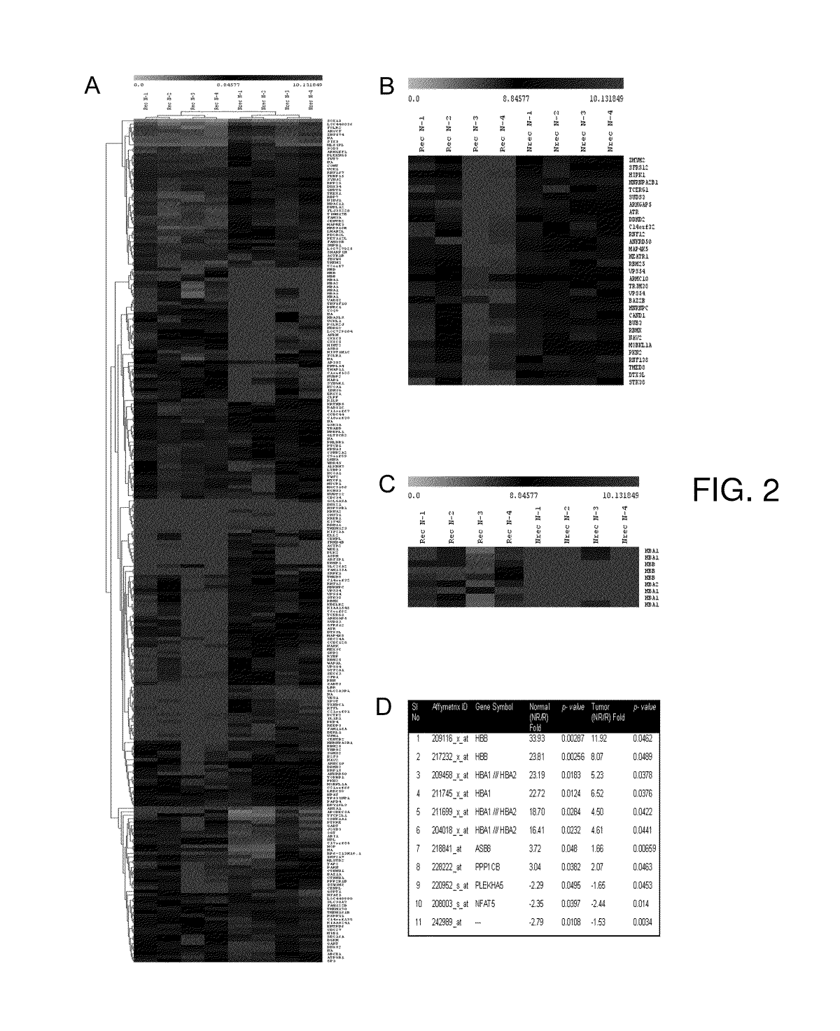

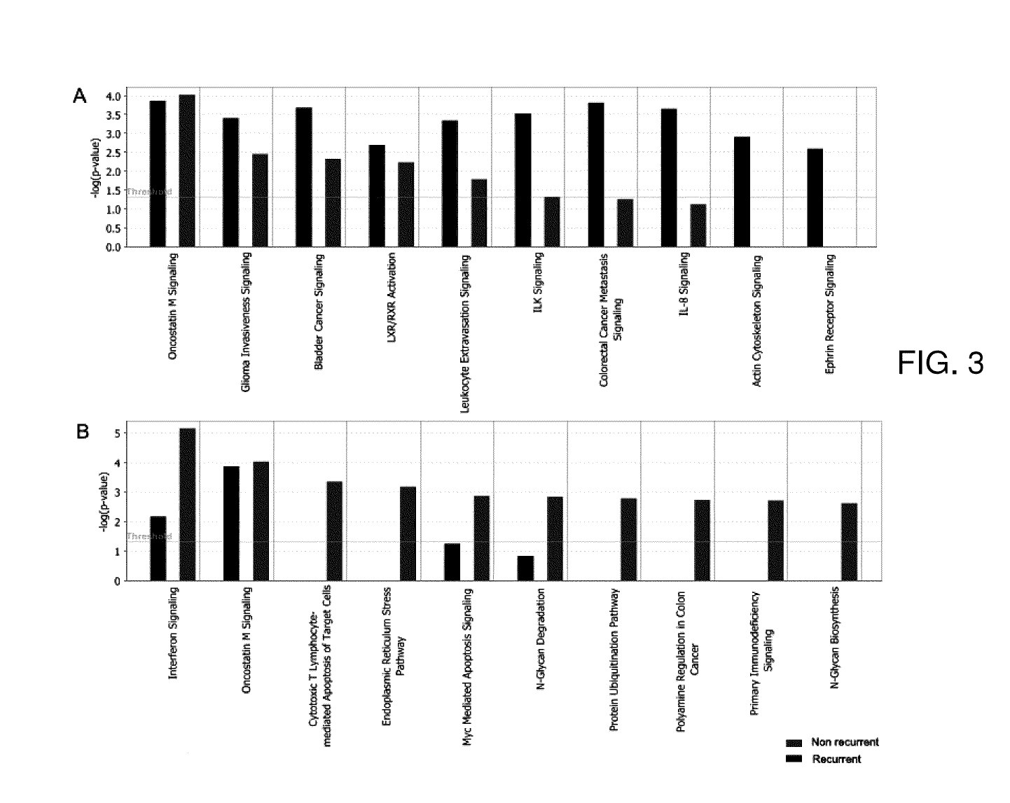

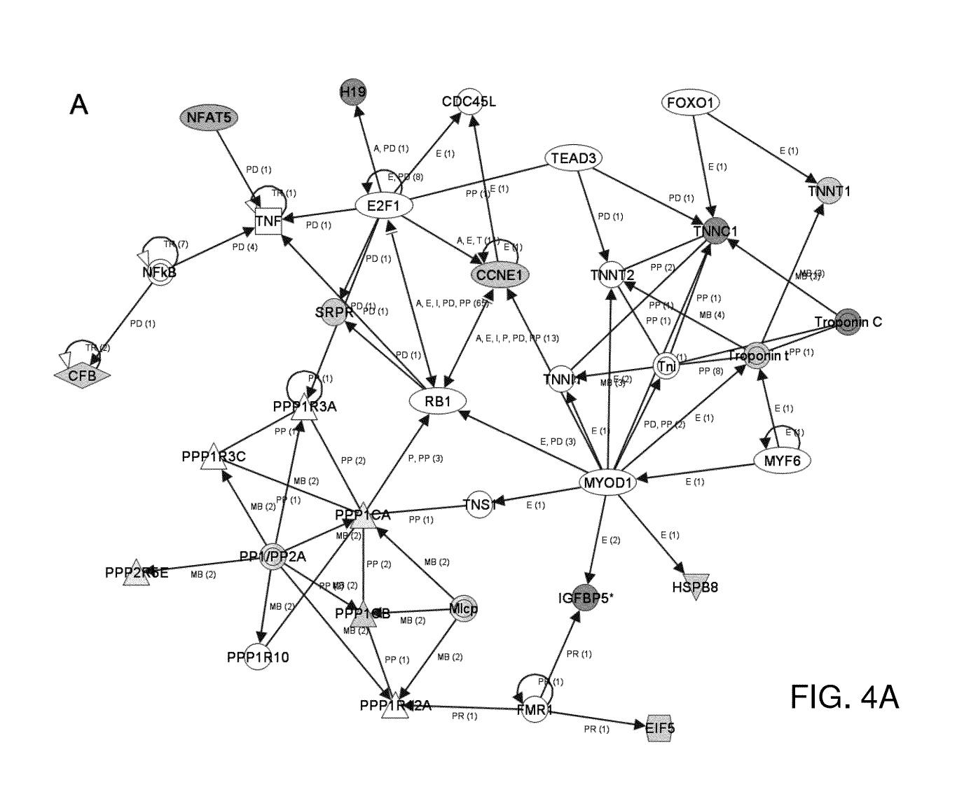

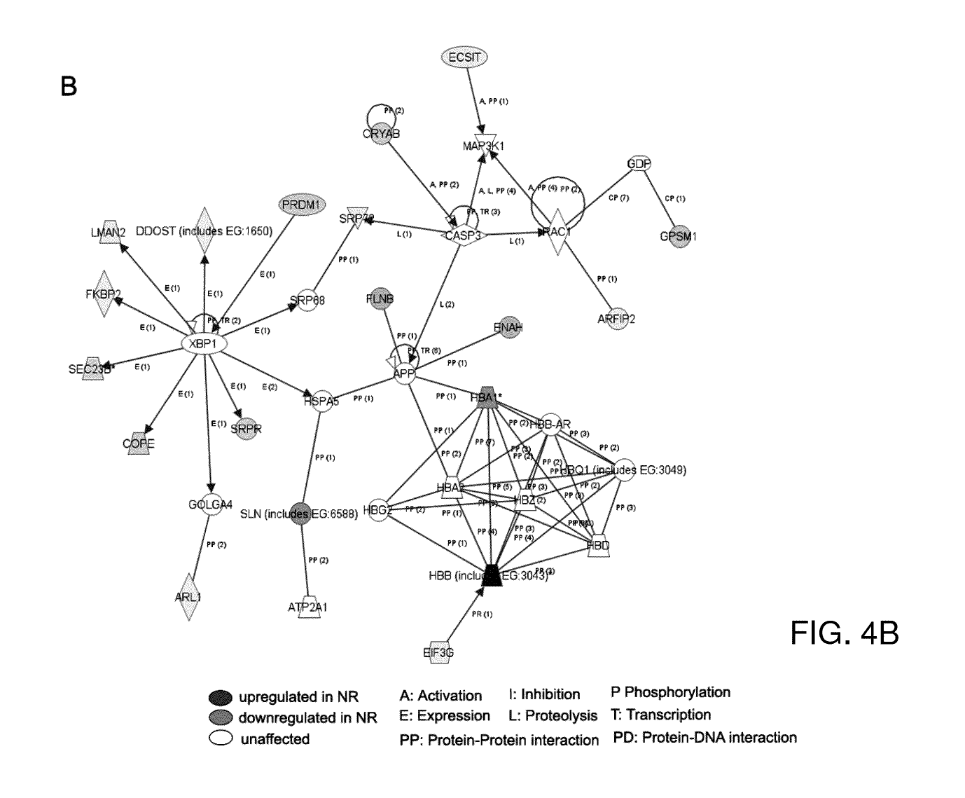

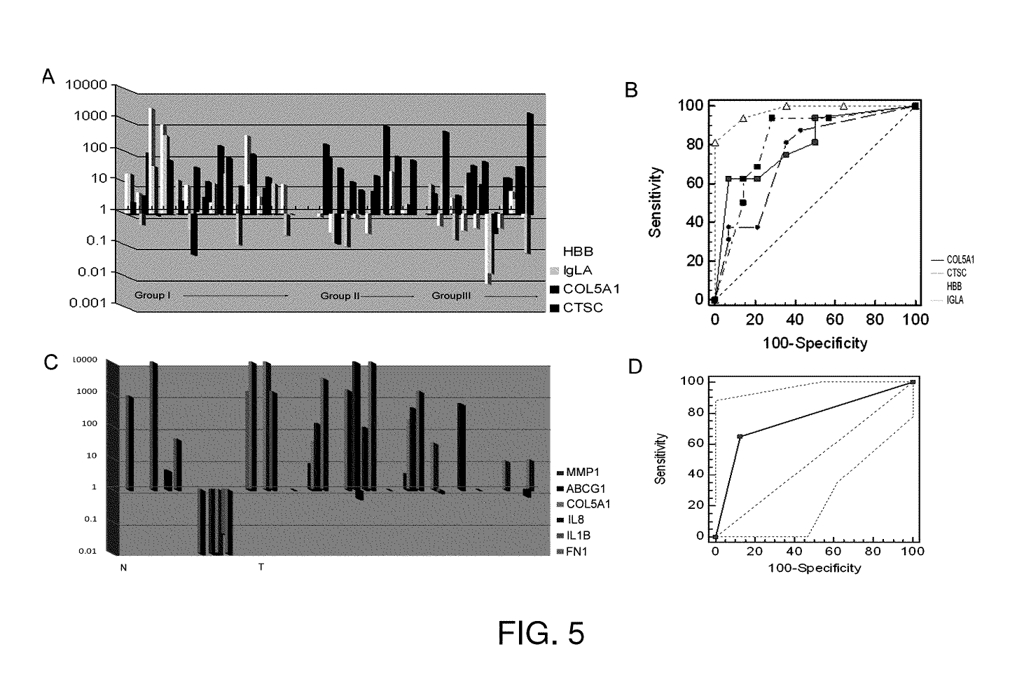

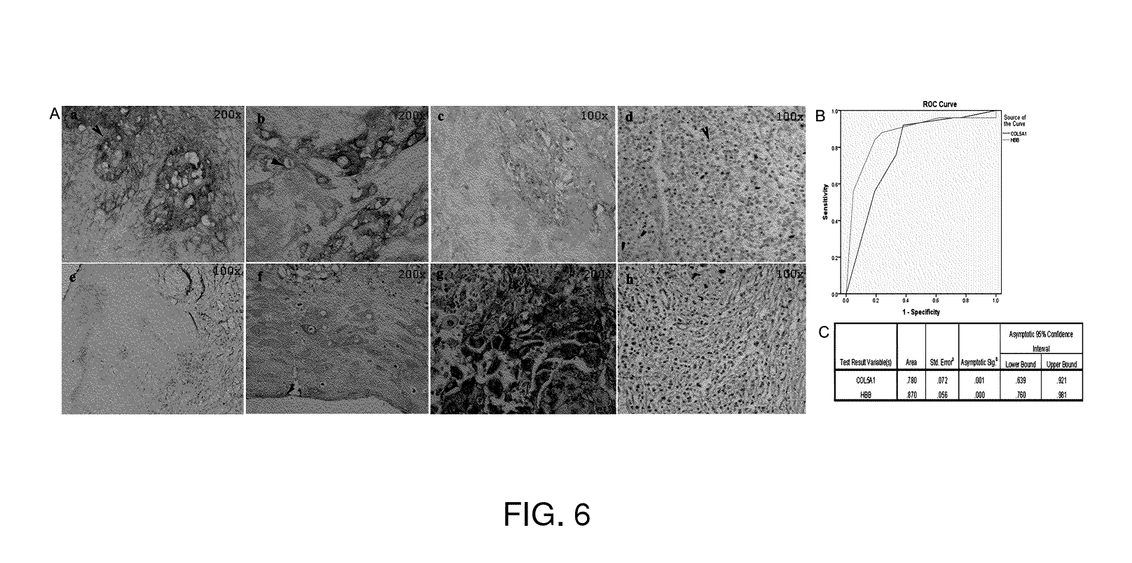

The present invention describes a molecular signature comprising of a set of genes or gene products whose altered expression in head and neck tumor in general including tongue cancer predicts (a) resistance to chemotherapy, which would help avoid chemotherapy or use other modalities of treatment (c) probability of recurrence of the disease post treatment (d) determining probability of metastasis at the time of surgery thereby allowing one to determine if adjuvant therapy is required or not.

The general molecular and cell biology methods used in this invention are known to those skilled in the art.

### EXAMPLES

In order that this invention be more fully understood the following preparative and testing examples are set forth. These examples are for the purpose of illustration only and are not to be construed as limiting the scope of the invention in any way. The examples described in this invention uses squamous cell carcinoma (tongue) as an example of head and neck cancer and other cancers, particularly carcinomas, and the invention and examples are generally applicable to all head and neck cancers as well other cancers, in particular carcinomas, as the genes and proteins involved in the molecular signature are common to cancer, hence would be generally applicable to most or all of these cancers.

### Example 1

### Patient Details and Sample Collection

The tissue samples are collected from patients undergoing surgical treatment after obtaining mandatory approvals (Table VI). The samples that were subjected to microarray analysis were collected in RNA later (Ambion, Austin, USA), while the samples for validation were either snap frozen or collected in RNA later and archived at −80° C. if required to be stored. The clinical characteristics of the patients are obtained from the electronic medical records maintained at the tertiary care cancer center. The sample sets were grouped into three categories: Group I (Pre-treatment, non-recurrent), which included pre-treatment tissues from patients who remained disease-free after standard treatment (surgery and adjuvant chemo radiation); Group II (Pre-treatment resistant/recurrent) included pre-treatment tissues from those who recurred during a 2-year follow up period; Group III (post-treatment recurrent; standard treatment) included recurrent tissue from patients with the recurrent disease. Group I & III were analyzed by micro array, while the validation was carried out in all the three groups. The adjacent mucosal tissue was collected 2 cm away from the tumor and confirmed as histologically negative for malignancy. Normal oral mucosa was also collected from non-diseased controls (age and risk factor matched) after written informed consent. Saliva samples were collected from healthy volunteers and previously untreated patients diagnosed with oral cancer (Stage I/II), after informed written consent. Unstimulated saliva was collected and mixed with RNAlater (Ambion, Austin USA) and stored at −80° C.

### Example 2

### RNA Isolation, Labeling of cRNA and Hybridization

Total RNA was isolated using the Qiagen RNeasy Kit (Qiagen, CA, US) and the samples that qualified through standard quality control criteria were selected for microarray. 100-200 ng of RNA was taken and biotinylated cRNA was prepared using the Two-cycle labeling Kit protocol (Affymetrix, CA, USA). The labeled cRNA was purified by the Genechip sample cleanup module (Qiagen, CA, US), fragmented and 20 μg hybridized to HGU133 plus 2 arrays (54,675 probes) using standard Affymetrix protocols. The hybridized chips were washed, stained and scanned by the Affymetrix Fluidics Station and Genechip Scanner 3000 using prescribed protocols.

### Example 3

### Microarray Analysis

The preliminary analysis to ascertain the internal controls and the hybridization efficiency was carried out using the Gene Chip Operating Software (GCOS) and Microarray Suite (MAS5, Affymetrix, CA, USA). The CEL files were extracted and imported into GeneSpring 7.2 (Agilent Technologies, CA, USA) software package for analysis. Raw image data were background corrected, normalized and summarized into probe set expression values using Robust Microarray Analysis (RMA) algorithm. For inter-array comparisons, data from each chip was normalized to 50% of the measurements taken from that chip (measurements of <0.01 were set to 0.01). Probe sets that were not reliably detected were removed, by filtering out those whose expression level was not >50 and confidence p-values <0.05, in at least 20% of the samples. To identify genes differentially expressed, both in the non-recurrent and recurrent tongue cancers as compared to adjacent mucosal samples, the remaining genes were subjected to Welch's t-test, not assuming variances equal, at p<0.05 and furthered filtered for fold change >1.5. Expression levels for individual genes are inferred as A) Differentially expressed genes identified in case of comparison with normal sample by measuring fold change (Fold change >2) or B) When only tumor samples are being analyzed, expression levels along with associated statistical significance values (p>0.01) are considered and these values are further normalized to a set of standard housekeeping genes. To determine differential Gene expression, samples were grouped into Normal/Tumor, recurrent and non-recurrent. 110 genes were differentially regulated in all the tumor samples (p<0.05), 212 in non-recurrent tumors (p<0.005) and 112 in recurrent tumors (p≦0.01) (Tables I, II & III respectively).

Ingenuity Pathway Analysis was carried out to identify significant functions, signaling pathways and networks (Ingenuity Systems Inc. CA, USA) at the default core analysis and core comparison platforms. Fishers exact test was used to identify the statistically significant functions/pathways. The differentially expressed genes were hierarchically clustered using Multi Experiment Viewer, v 4.5 (MeV) (TM4 Microarray Software Suite, The Institute of Genomic Research (TIGR) with the Euclidean distance measurement and p values were calculated after application of the non-parametric Wilcoxon-Mann Whitney test (p<0.5). Furthermore, K-means clustering (K=10; Euclidean distance) was carried out to identify a sub-set of genes that would clearly differentiate the groups under study.

### Example 4

### Validation of the Microarray Data in Tissue and Saliva Samples by Quantitative PCR

RNA was isolated from tissues using Tri Reagent (Sigma Aldrich, MO, USA), first strand synthesis was done using MMLV Reverse transcriptase (Ambion, Austin, USA) and Quantitative Real Time PCR (QRT PCR) by the Power Syber Green kit (Applied Biosystems, CA, USA) in an ABI 7300 Cycler (Applied Biosystems, CA, USA). The expression levels of the genes selected for validation (MMP1, EMP1, ABCG1, COL5A1, IgLA, HBB, CTSC and CCL18) (Table I) was assessed by QRT PCR using the relative quantification (ΔΔCT method). Expression was normalized using the endogenous control (GAPDH) and normal oral mucosal tissues were used as the calibrator. Melting curve analysis was done to ensure the specificity of the product obtained.

Unstimulated saliva collected from patients/controls was mixed with RNAlater; subsequently the samples were centrifuged at 14,000 rpm for 20 minutes at 4° C. RNA was isolated from the salivary supernatant using the Qiagen Viral RNA Kit (Qiagen, CA, US). The samples were assessed for their integrity using the expression of the endogenous control (GAPDH) by Reverse Transcription PCR (RT-PCR) as a criterion. A subset of 10 candidate markers (MMP1, FN1, FAPA, SERPINH2, IL8, IL1B, IgLA, ABCG1, COL5A1, HBB), were tested for their expression in saliva by QRT PCR as above. Saliva samples from healthy volunteers as the calibrator. The detection of one or more markers in the samples was considered as ‘test positive=1’ while absence of any of the markers was considered ‘test negative=0’. The combined test result in the binary input format was used for the statistical analysis. The expression patterns were correlated to the disease status of the patients to ascertain their clinical relevance.

### Example 5

### Immunohistochemical Analysis

The protein expression of two representative genes (COL5A1 and HBB), validated by QRT PCR was profiled in the tissue sections of a different cohort of patients with tongue cancer. The sections were deparaffinized and IHC carried out according to standard protocols. The antibodies were used in dilutions of 1:50 for both COL5A1 (sc133162; Santacruz Biotechnology, Santacruz, Calif., USA) and HBB (H4890; Sigma Aldrich, USA). The sections were microwaved for antigen retrieval and the staining detected by Dako REAL EnVision™ kit (Dako Corporation, Carpenteria, Calif., USA). The sections were counterstained using haematoxylin and scanned at low and high power to identify areas of even staining and percentage of positive cells. The grades of positivity were scored as follows; negative (<1%), grade I (1-10%), grade II (10-30%), III (30-60%) and IV (>60%). The intensity of staining was also graded as mild, moderate and intense. The expression in the normal oral mucosal tissues was used as control.

Receiver Operating Characteristic (ROC) curve analyses were carried out by SPSS 19 (IBM) and MedCalc® v 11.6.0.0 for the QPCR and IHC results. Area under the curve was computed via numerical integration of the ROC curves. The biomarkers, individually or in combination, with the largest Area under Curve (AUC) were identified to have the maximum predictive power for disease recurrence. Multiple regression analysis was also carried out by the stepwise method to identify the predictive value of the marker combinations.

### Example 6

### Determination of Molecular Signature from FFPE Samples

Formalin-fixed paraffin embedded (FFPE) samples of tumor and adjacent tissue is a convenient source for obtaining RNA for identification of the molecular signature described in this invention.

10 μm curl sections is cut from FFPE blocks of cancer or adjacent tissue, placed in a 1.5 ml micro centrifuge tube and heated at 70° C. in a heating block for 20 min to allow excess paraffin wax to be removed. Pre-warmed xylene (1 ml) is added to the tube and heated at 50° C. for 10 min. The microfuge tube is then centrifuged at 12000 g for 2 min in a micro centrifuge. Waste xylene is removed by pipette and the xylene wash repeated twice more. Residual xylene is removed by the addition of 1.0 ml of 100% ethanol to the dewaxed tissue sections, which will be allowed to stand for 10 min at room temperature. The tissue is centrifuged 12,000 g for 5 min and the ethanol removed by pipette, and the process repeated once more with 100% ethanol. The tissue is rehydrated with 1.0 ml 90% ethanol for 5 min and finally washed in 1.0 ml 70% ethanol for 5 min. The sample is air dried to allow the ethanol to evaporate completely prior to protease digestion.

Protease digestion is performed by use of a Recoverall Kit™ (Applied Biosystems, AM1975) as per the manufacturer's protocol following which 480 μl of the Ambion RecoverAll™ Isolation Additive is added to the microfuge tube, and vortex mixed for 20 seconds and allowed to stand for 15 min at room temperature. The tubes are pulse spun in a microfuge at 12000 g for 30 seconds. Two 240 μl aliquots of the resulting lysate is then stored at −20° C. for RNA extraction.

RNA extraction is performed using the Recoverall Kit™ as per manufacturer's instructions. RNA is eluted finally in a volume of 60 μl. Purity and quantity are checked spectrophotometry at 260 nm and 280 nm by placing 1.3 μl of eluate on the sampling pedestal of a scanning spectrophotometer. Aliquots of each sample are stored at −80° C. or reverse transcribed to produce cDNA in a two step RT-PCR reaction. RNA from fresh-frozen samples will be obtained using the RNeasy kit from Qiagen, according to the manufacturer's protocol.

The amount and quality of RNAs is assessed by UV spectrophotometry and considered adequate for further analysis if the optical density 260/280 ratio is >1.8 and the total RNA yield >500 ng.

Preparation of cDNA

Reverse transcription is performed using an ABI High-Capacity cDNA Archive Kit according to the manufacturer's instructions. cDNA content is measured using a spectrophotometer. In the case of RNA-cDNA from FFPE tissues, PCRs of a housekeeper gene (e.g. PGK) with amplicons of increasing length (from 50 to 200) is run on a 3% agarose gel to check the distribution of fragment lengths.

Polymerase Chain Reaction

Quantitative Real Time PCR (QRT PCR) is carried out by the SYBR Green or Fluorescent dual labeled probe method on a real-time PCR machine, in this case—an ABI 7300 Cycler (Applied Biosystems, CA, USA). The expression levels of the genes selected from Table X are assessed by QRT PCR using either the relative quantification method (ΔΔCT method) [Livak and Schmittgen, Methods 25 (2001), 402-408] using normalizer genes such as GAPDH, which is used in the present study. Normal oral mucosal tissue or other standard RNA samples could be used as Calibrator, if required. Melting curve analysis is done to ensure the specificity of the product obtained, when using SYBR green method.

### Example 7

### Interpretation of Molecular Signature

Molecular signature can be identified by determining the expression of the individual genes represented in the signature or through determination of the proteins that these genes encode. While several methods can be used to determine the molecular signature identified in this invention, the following method is used to draw inferences from the molecular signature based on values in Table X as follows
1. A poor prognosis indicating recurrence/metastasis/failure of chemotherapy, radiation therapy or other therapies are indicated if high expression levels are seen for majority of genes listed at no. 1-19 and 47-50. At the same time absence/low expression for majority of genes listed at no. 20-29; 30-46 and 51-108 will corroborate the inference
2. A good prognosis indicating non-recurrence/absence of metastasis/response to chemotherapy radiation therapy or other therapies are indicated if high expression levels are seen for majority of genes listed at no. 30-46 and 51-108. At the same time absence/low expression for majority of genes listed at no. 1-19; 47-50 and 109 will corroborate the inference

### Tables

TABLE I
Differentially expressed genes in the oral tongue tumors (p < 0.05)

| S | Affymetrix | Gene | | Fold | p | Fold | Fold |
|---|---|---|---|---|---|---|---|
| NO | ID | Symbol | p (R/Normal)* | (R/Normal) | (NR/Normal)# | (NR/Normal) | Diff$ |
| 1 | 204567_s_at | ABCG1 | 3.83E−05 | 6.71 | 0.00166 | 3.78 | 2.93 |
| 2 | 204169_at | IMPDH1 | 0.00234 | 1.95 | 0.0351 | 2.11 | −0.16 |
| 3 | 205479_s_at | PLAU | 0.00409 | 7.66 | 0.00268 | 4.95 | 2.70 |
| 4 | 204475_at | MMP1 | 0.00519 | 74.50 | 0.00012 | 255.50 | −181.0 |
| 5 | 202897_at | SIRPA | 0.00538 | 3.31 | 0.02 | 3.11 | 0.21 |
| 6 | 203417_at | MFAP2 | 0.00596 | 5.40 | 0.00102 | 5.44 | −0.04 |
| 7 | 225898_at | WDR54 | 0.00674 | 3.13 | 0.00106 | 3.19 | −0.06 |
| 8 | 227484_at | — | 0.00692 | 2.17 | 0.00872 | 2.87 | −0.70 |
| 9 | 221538_s_at | PLXNA1 | 0.00821 | 3.56 | 0.0117 | 2.59 | 0.98 |
| 10 | 203562_at | FEZ1 | 0.00837 | 6.14 | 0.036 | 3.20 | 2.94 |
| 11 | 224472_x_at | SDF4 | 0.00962 | 1.69 | 0.0459 | 1.79 | −0.11 |
| 12 | 221523_s_at | RRAGD | 0.0102 | −4.00 | 0.0103 | −5.03 | 1.03 |
| 13 | 207714_s_at | SERPINH1 | 0.0109 | 3.18 | 0.00855 | 3.97 | −0.80 |
| 14 | 204924_at | TLR2 | 0.0118 | 3.32 | 0.00355 | 3.05 | 0.26 |
| 15 | 205828_at | MMP3 | 0.0141 | 26.15 | 0.000288 | 35.40 | −9.25 |
| 16 | 218089_at | C20orf4 | 0.0142 | 1.60 | 0.00069 | 1.58 | 0.02 |
| 17 | 221898_at | PDPN | 0.0148 | 5.97 | 0.0022 | 5.74 | 0.23 |
| 18 | 205680_at | MMP10 | 0.0151 | 23.70 | 0.00102 | 29.51 | −5.81 |
| 19 | 204214_s_at | RAB32 | 0.0158 | 2.37 | 0.00044 | 2.27 | 0.10 |
| 20 | 218847_at | IGF2BP2 | 0.0159 | 3.56 | 0.00146 | 3.32 | 0.24 |
| 21 | 212740_at | PIK3R4 | 0.0171 | 1.76 | 0.0041 | 1.61 | 0.14 |
| 22 | 217196_s_at | CAMSAP1L1 | 0.0172 | 1.61 | 0.0196 | 3.56 | −1.95 |
| 23 | 221730_at | COL5A2 | 0.0179 | 7.79 | 0.014 | 7.00 | 0.78 |
| 24 | 204140_at | TPST1 | 0.0182 | 3.33 | 0.0112 | 3.17 | 0.16 |
| 25 | 223095_at | MARVELD1 | 0.0186 | 2.10 | 0.0324 | 1.52 | 0.58 |
| 26 | 55093_at | CSGlcA-T | 0.0191 | 2.18 | 0.0091 | 2.47 | −0.29 |
| 27 | 225285_at | BCAT1 | 0.0196 | 6.16 | 0.0265 | 3.98 | 2.18 |
| 28 | 212488_at | COL5A1 | 0.0197 | 7.18 | 0.0117 | 5.88 | 1.30 |
| 29 | 225401_at | C1orf85 | 0.0202 | 2.21 | 0.0048 | 2.56 | −0.35 |
| 30 | 205959_at | MMP13 | 0.0205 | 25.45 | 0.0313 | 10.91 | 14.54 |
| 31 | 202458_at | PRSS23 | 0.0205 | 4.53 | 0.000186 | 8.77 | −4.24 |
| 32 | 202998_s_at | LOXL2 | 0.0206 | 5.31 | 0.0452 | 3.69 | 1.62 |
| 33 | 203936_s_at | MMP9 | 0.0206 | 8.39 | 0.00438 | 13.60 | −5.22 |
| 34 | 225205_at | KIF3B | 0.0208 | 1.55 | 0.0101 | 1.92 | −0.36 |
| 35 | 227846_at | GPR176 | 0.0209 | 5.00 | 0.00381 | 4.00 | 1.00 |
| 36 | 201954_at | ARPC1B | 0.0209 | 2.65 | 0.00383 | 2.71 | −0.05 |
| 37 | 202369_s_at | TRAM2 | 0.0209 | 2.39 | 0.0254 | 3.50 | −1.11 |
| 38 | 204041_at | MAOB | 0.0217 | −5.08 | 0.00502 | −4.39 | −0.69 |
| 39 | 202391_at | BASP1 | 0.0219 | 3.41 | 0.0265 | 6.22 | −2.81 |
| 40 | 213139_at | SNAI2 | 0.0222 | 2.81 | 0.00014 | 5.81 | −3.00 |
| 41 | 200618_at | LASP1 | 0.0223 | 1.84 | 0.015 | 1.87 | −0.03 |
| 42 | 203066_at | GALNAC4S- | 0.0224 | 2.66 | 0.0234 | 2.77 | −0.11 |
| | | 6ST | | | | | |
| 43 | 204137_at | GPR137B | 0.0227 | 2.16 | 0.0142 | 3.82 | −1.66 |
| 44 | 228273_at | — | 0.0235 | 2.54 | 0.0296 | 7.10 | −4.55 |
| 45 | 226609_at | DCBLD1 | 0.0239 | 3.60 | 0.0222 | 4.12 | −0.52 |
| 46 | 209166_s_at | MAN2B1 | 0.024 | 1.87 | 0.00842 | 2.54 | −0.67 |
| 47 | 222108_at | AMIGO2 | 0.024 | 3.27 | 0.00224 | 5.25 | −1.99 |
| 48 | 223507_at | CLPX | 0.0246 | −1.55 | 0.0143 | −1.86 | 0.32 |
| 49 | 218196_at | OSTM1 | 0.0246 | 2.36 | 0.0113 | 2.35 | 0.01 |
| 50 | 214297_at | CSPG4 | 0.0249 | 5.44 | 0.0144 | 4.18 | 1.26 |
| 51 | 202727_s_at | IFNGR1 | 0.0253 | 1.98 | 0.00783 | 2.17 | −0.20 |
| 52 | 209934_s_at | ATP2C1 | 0.0256 | 2.39 | 0.00824 | 2.27 | 0.12 |
| 53 | 203879_at | PIK3CD | 0.0256 | 2.28 | 0.00574 | 2.69 | −0.42 |
| 54 | 203038_at | PTPRK | 0.026 | 2.39 | 0.0473 | 1.74 | 0.65 |
| 55 | 218224_at | PNMA1 | 0.0267 | 2.66 | 0.0201 | 2.37 | 0.29 |
| 56 | 241353_s_at | — | 0.0271 | 1.93 | 0.0143 | 1.72 | 0.21 |
| 57 | 203505_at | ABCA1 | 0.0273 | 2.21 | 0.00302 | 2.58 | −0.37 |
| 58 | 203650_at | PROCR | 0.0275 | 2.83 | 0.0097 | 2.65 | 0.19 |
| 59 | 224735_at | CYBASC3 | 0.028 | 1.91 | 0.0292 | 1.79 | 0.12 |
| 60 | 214853_s_at | SHC1 | 0.0283 | 2.72 | 0.00195 | 2.47 | 0.24 |
| 61 | 207643_s_at | TNFRSF1A | 0.0283 | 1.66 | 0.0366 | 1.61 | 0.04 |
| 62 | 223107_s_at | ZCCHC17 | 0.0288 | 1.74 | 0.0165 | 1.59 | 0.15 |
| 63 | 219684_at | RTP4 | 0.0292 | 3.25 | 0.0034 | 3.53 | −0.28 |
| 64 | 218130_at | C17orf62 | 0.0294 | 2.56 | 0.00845 | 2.94 | −0.38 |
| 65 | 218404_at | SNX10 | 0.0297 | 3.31 | 0.00437 | 4.40 | −1.09 |
| 66 | 32069_at | N4BP1 | 0.03 | 1.76 | 0.0356 | 2.66 | −0.89 |
| 67 | 214329_x_at | TNFSF10 | 0.0303 | 4.09 | 0.0312 | 2.96 | 1.13 |
| 68 | 223463_at | RAB23 | 0.0305 | 2.22 | 0.0464 | 2.18 | 0.04 |
| 69 | 208012_x_at | SP110 | 0.0307 | 2.10 | 0.00774 | 2.50 | −0.40 |
| 70 | 218968_s_at | ZFP64 | 0.031 | 1.61 | 0.0101 | 1.69 | −0.08 |
| 71 | 226682_at | LOC283666 | 0.031 | −2.85 | 0.0128 | −2.48 | −0.37 |
| 72 | 205324_s_at | FTSJ1 | 0.0312 | 1.78 | 0.0206 | 2.03 | −0.25 |
| 73 | 225646_at | CTSC | 0.0319 | 4.66 | 0.0058 | 7.17 | −2.51 |
| 74 | 203764_at | DLG7 | 0.0321 | 2.04 | 0.0496 | 7.82 | −5.78 |
| 75 | 209684_at | RIN2 | 0.0327 | 1.77 | 0.00513 | 2.27 | −0.51 |
| 76 | 225076_s_at | ZNFX1 | 0.0328 | 1.78 | 0.0279 | 1.84 | −0.06 |
| 77 | 229450_at | IFIT3 | 0.0331 | 4.07 | 0.0172 | 5.58 | −1.52 |
| 78 | 201976_s_at | MYO10 | 0.0333 | 2.21 | 0.00396 | 3.96 | −1.75 |
| 79 | 219522_at | FJX1 | 0.0342 | 2.60 | 0.0333 | 3.91 | −1.31 |
| 80 | 225636_at | STAT2 | 0.0345 | 2.02 | 0.0311 | 2.01 | 0.01 |
| 81 | 202859_x_at | IL8 | 0.0352 | 7.67 | 0.0129 | 13.10 | −5.43 |
| 82 | 204000_at | GNB5 | 0.0356 | 2.17 | 0.0495 | 1.64 | 0.53 |
| 83 | 218154_at | GSDMDC1 | 0.037 | 1.79 | 0.0278 | 1.70 | 0.09 |
| 84 | 203381_s_at | APOE | 0.0371 | 2.51 | 0.0105 | 2.16 | 0.35 |

TABLE II
Differentially expressed genes in the non-
recurrent oral tongue tumors (p < 0.05)

| Sl | | | | |
|---|---|---|---|---|
| No | Affymetrix ID | P-value | Fold | Gene Symbol |
| 1 | 204475_at | 0.00012 | 255.50 | MMP1 |
| 2 | 204580_at | 0.000692 | 64.16 | MMP12 |
| 3 | 214677_x_at | 0.00367 | 50.26 | MEF2A |
| 4 | 211430_s_at | 0.00483 | 41.16 | IGH@ /// |
| | | | | IGHG1 /// |
| | | | | IGHG2 /// |
| | | | | IGHG3 /// |
| | | | | IGHM /// |
| | | | | IGHV4-31 |
| 5 | 209138_x_at | 0.00186 | 36.17 | IGL@ |
| 6 | 205828_at | 0.000288 | 35.40 | MMP3 |
| 7 | 205680_at | 0.00102 | 29.51 | MMP10 |
| 8 | 201645_at | 0.000184 | 28.77 | TNC |
| 9 | 211756_at | 0.000497 | 28.52 | PPIA |
| 10 | 215121_x_at | 0.00254 | 27.28 | PABPC1 |
| 11 | 209395_at | 0.00282 | 24.94 | CHI3L1 |
| 12 | 215379_x_at | 0.00111 | 24.04 | LOX |
| 13 | 209924_at | 0.000224 | 21.57 | CCL18 |
| 14 | 202267_at | 0.00441 | 16.25 | LAMC2 |
| 15 | 225681_at | 0.00454 | 16.01 | FAM33A |
| 16 | 1556773_at | 0.00041 | 15.18 | — |
| 17 | 218468_s_at | 0.000843 | 14.26 | GREM1 |
| 18 | 32128_at | 0.000984 | 13.70 | TREX1 |
| 19 | 203936_s_at | 0.00438 | 13.60 | MMP9 |
| 20 | 210355_at | 0.000551 | 13.36 | PTHLH |
| 21 | 221671_x_at | 0.00132 | 13.29 | CLEC7A |
| 22 | 221651_x_at | 0.00283 | 13.19 | ARHGEF10L |
| 23 | 204533_at | 0.00187 | 11.34 | CXCL10 |
| 24 | 215446_s_at | 0.000434 | 10.80 | SEC16A |
| 25 | 204415_at | 0.00471 | 9.75 | IFI6 |
| 26 | 225647_s_at | 7.29E−05 | 9.66 | UHRF1 |
| 27 | 203915_at | 0.00128 | 9.54 | CXCL9 |
| 28 | 227609_at | 0.00216 | 9.10 | LOC493869 |
| 29 | 202458_at | 0.000186 | 8.77 | PRSS23 |
| 30 | 206513_at | 0.000704 | 8.65 | AIM2 |
| 31 | 206026_s_at | 0.000441 | 7.44 | TNFAIP6 |
| 32 | 205159_at | 0.00094 | 6.79 | CSF2RB |
| 33 | 212314_at | 0.00475 | 6.61 | TMED10 |
| 34 | 201422_at | 0.000631 | 6.50 | IFI30 |
| 35 | 212364_at | 7.84E−05 | 6.38 | MYO1B |
| 36 | 201579_at | 0.000503 | 6.37 | FAT |
| 37 | 207039_at | 0.0043 | 6.30 | CDKN2A |
| 38 | 225639_at | 0.00148 | 5.83 | C14orf32 |
| 39 | 213139_at | 0.00014 | 5.81 | SP3 |
| 40 | 226368_at | 0.000587 | 5.74 | CHST11 |
| 41 | 221898_at | 0.0022 | 5.74 | CYLD |
| 42 | 226279_at | 0.00366 | 5.65 | FAM91A1 |
| 43 | 209360_s_at | 0.000443 | 5.55 | RUNX1 |
| 44 | 203417_at | 0.00102 | 5.44 | MFAP2 |
| 45 | 229400_at | 0.0015 | 5.44 | IFIT3 |
| 46 | 222108_at | 0.00224 | 5.25 | GPR172A |
| 47 | 203423_at | 0.00155 | 5.25 | RBP1 |
| 48 | 212588_at | 0.00348 | 5.19 | RRAS2 |
| 49 | 221059_s_at | 0.00174 | 5.15 | TXNDC5 |
| 50 | 204972_at | 0.00295 | 5.15 | OAS2 |
| 51 | 204337_at | 0.00454 | 5.13 | RGS4 |
| 52 | 203313_s_at | 0.0036 | 5.05 | TGIF1 |
| 53 | 218400_at | 0.003 | 5.05 | SNX10 |
| 54 | 202953_at | 0.000743 | 5.01 | C1QB |
| 55 | 205479_s_at | 0.00268 | 4.95 | PLAU |
| 56 | 212365_at | 0.00127 | 4.77 | GART |
| 57 | 204222_s_at | 0.000446 | 4.65 | GLIPR1 |
| 58 | 201487_at | 0.00284 | 4.52 | CTSC |
| 59 | 202558_s_at | 0.000662 | 4.50 | STCH |
| 60 | 201564_s_at | 0.00094 | 4.45 | FSCN1 |
| 61 | 206584_at | 0.000407 | 4.44 | LY96 |
| 62 | 218404_at | 0.00437 | 4.40 | NDE1 |
| 63 | 201853_s_at | 0.00253 | 4.35 | CDC25B |
| 64 | 203083_at | 0.00134 | 4.34 | THBS2 |
| 65 | 201818_at | 0.000494 | 4.34 | LPCAT1 |
| 66 | 226621_at | 0.000208 | 4.30 | LOC401504 |
| 67 | 204362_at | 0.000204 | 4.29 | SKAP2 |
| 68 | 201417_at | 0.00397 | 4.20 | SOX4 |
| 69 | 221881_s_at | 0.000439 | 4.19 | PDPN |
| 70 | 226372_at | 0.000739 | 4.18 | ERGIC2 |
| 71 | 200644_at | 0.00221 | 4.10 | MARCKSL1 |
| 72 | 208966_x_at | 0.00496 | 4.05 | IFI16 |
| 73 | 227846_at | 0.00381 | 4.00 | FAM125A |
| 74 | 210164_at | 0.00115 | 3.96 | GZMB |
| 75 | 201976_s_at | 0.00396 | 3.96 | MYO10 |
| 76 | 202357_s_at | 0.00261 | 3.92 | CFB |
| 77 | 209476_at | 0.0024 | 3.87 | TXNDC1 |
| 78 | 203476_at | 0.000953 | 3.86 | TPBG |
| 79 | 200698_at | 0.00427 | 3.84 | KDELR2 |
| 80 | AFFX- | 0.000732 | 3.83 | bioB |
| | HUMISGF3A/ | | | |
| | M97935_3_at | | | |
| 81 | 204567_s_at | 0.00166 | 3.78 | ABCG1 |
| 82 | 223343_at | 0.000679 | 3.73 | C6orf115 |
| 83 | 218699_at | 0.00111 | 3.72 | NXT1 |
| 84 | 201720_s_at | 0.00279 | 3.68 | LAPTM5 |
| 85 | 217892_s_at | 0.000594 | 3.68 | C1orf108 |
| 86 | 225258_at | 0.000254 | 3.64 | RBMS1 |
| 87 | 223158_s_at | 0.000975 | 3.62 | RHOU |
| 88 | 229860_x_at | 0.00412 | 3.59 | CLCC1 |
| 89 | 202820_at | 0.00129 | 3.55 | AHR |
| 90 | 201669_s_at | 0.00326 | 3.54 | MARCKS |
| 91 | 219684_at | 0.0034 | 3.53 | APOL6 |
| 92 | 200989_at | 0.00393 | 3.50 | HIF1A |
| 93 | 201088_at | 0.00443 | 3.50 | KPNA2 |
| 94 | 208103_s_at | 0.00302 | 3.46 | ANP32E |
| 95 | 200599_s_at | 0.00342 | 3.46 | HSP90B1 |
| 96 | 218847_at | 0.00146 | 3.32 | NETO2 |
| 97 | 219434_at | 0.00169 | 3.29 | EGFL6 |
| 98 | 238725_at | 0.00333 | 3.26 | — |
| 99 | 200755_s_at | 0.000661 | 3.23 | CALU |
| 100 | 202666_s_at | 0.00185 | 3.22 | ACTL6A |
| 101 | 226756_at | 0.00146 | 3.22 | — |
| 102 | 214456_x_at | 0.00163 | 3.21 | BCLAF1 |
| 103 | 225415_at | 0.00125 | 3.20 | GTF2A1 |
| 104 | 202088_at | 0.00429 | 3.20 | SLC39A6 |
| 105 | 225898_at | 0.00106 | 3.19 | TP53INP1 |
| 106 | 222690_s_at | 0.00484 | 3.18 | FNDC3B |
| 107 | 202720_at | 0.00465 | 3.14 | TES |
| 108 | 213287_s_at | 0.00339 | 3.13 | TRIM22 |
| 109 | 224793_s_at | 0.00127 | 3.12 | IGK@ /// IGKC |
| | | | | /// IGKV1-5 /// |
| | | | | IGKV2-24 |
| 110 | 218595_s_at | 0.00258 | 3.12 | DRAM |
| 111 | 221020_s_at | 0.00484 | 3.11 | CKLF |
| 112 | 218368_s_at | 0.00232 | 3.09 | AKTIP |
| 113 | 222457_s_at | 0.00315 | 3.08 | EFHD2 |
| 114 | 204092_s_at | 0.00318 | 3.06 | AURKA |
| 115 | 208637_x_at | 0.00183 | 3.06 | ACTN1 |
| 116 | 53720_at | 0.00176 | 3.05 | MICALL1 |
| 117 | 204924_at | 0.00355 | 3.05 | TLR2 |
| 118 | 201656_at | 0.00316 | 3.05 | ITGA6 |
| 119 | 231823_s_at | 0.000674 | 3.05 | ODZ2 |
| 120 | 200887_s_at | 0.00168 | 3.02 | STAT1 |
| 121 | 219161_s_at | 0.00298 | 3.00 | RHBDF2 |
| 122 | 202381_at | 0.00474 | 2.99 | ADAMS |
| 123 | 205443_at | 0.00148 | 2.97 | SNAPC1 |
| 124 | 201091_s_at | 0.000484 | 2.96 | CBX3 /// |
| | | | | LOC653972 |
| 125 | 201667_at | 0.00179 | 2.96 | GJA1 |
| 126 | 225439_at | 0.00278 | 2.92 | MIER1 |
| 127 | 207181_s_at | 1.73E−06 | 2.91 | CASP7 |
| 128 | 211676_s_at | 0.00101 | 2.88 | BID |
| 129 | 225731_at | 0.00479 | 2.84 | ETV6 |
| 130 | 225853_at | 0.00427 | 2.82 | TRIM47 |
| 131 | 1558693_s_at | 0.00298 | 2.77 | C1orf85 |
| 132 | 201649_at | 0.000545 | 2.77 | UBE2L6 |
| 133 | 203693_s_at | 0.004 | 2.76 | E2F3 |
| 134 | 1558080_s_at | 0.00489 | 2.76 | LOC144871 |
| 135 | 217776_at | 0.00275 | 2.73 | YKT6 |
| 136 | 209852_x_at | 0.00468 | 2.72 | PSME3 |
| 137 | 208689_s_at | 0.00287 | 2.71 | RPN2 |
| 138 | 201954_at | 0.00383 | 2.71 | ARPC1B /// |
| | | | | LOC653888 |
| 139 | 200839_s_at | 0.00208 | 2.70 | CTSB |
| 140 | 201128_s_at | 0.00174 | 2.69 | ACLY |
| 141 | 208918_s_at | 0.00329 | 2.67 | NADK |
| 142 | 201300_s_at | 0.00039 | 2.64 | PRNP |
| 143 | 208703_s_at | 0.00258 | 2.62 | APLP2 |
| 144 | 203505_at | 0.00302 | 2.58 | ABCA1 |
| 145 | 225401_at | 0.0048 | 2.56 | — |
| 146 | 201776_s_at | 0.00145 | 2.54 | KIAA0494 |
| 147 | 212063_at | 0.00481 | 2.51 | GPR56 |
| 148 | 213399_x_at | 0.00386 | 2.49 | MFHAS1 |
| 149 | 214853_s_at | 0.00195 | 2.47 | SFRS2 |
| 150 | 217813_s_at | 0.00385 | 2.47 | ENAH |
| 151 | 213491_x_at | 0.00484 | 2.46 | ADAM17 |
| 152 | 219540_at | 0.00386 | 2.44 | EAF2 |
| 153 | 224753_at | 0.00453 | 2.41 | PAFAH1B2 |
| 154 | 202603_at | 0.00146 | 2.41 | — |
| 155 | 201944_at | 0.00128 | 2.41 | HEXB |
| 156 | 208674_x_at | 0.00369 | 2.40 | DDOST |
| 157 | 206976_s_at | 0.00422 | 2.36 | HSPH1 |
| 158 | 201761_at | 0.00094 | 2.35 | MTHFD2 |
| 159 | 223451_s_at | 0.00488 | 2.33 | CXCL16 |
| 160 | 225479_at | 0.004 | 2.31 | FRMD6 |
| 161 | 226893_at | 0.00487 | 2.30 | LRIG3 |
| 162 | 204214_s_at | 0.00044 | 2.27 | RAB32 |
| 163 | 200902_at | 0.0033 | 2.26 | Sep15 |
| 164 | 202059_s_at | 0.00194 | 2.24 | KPNA1 |
| 165 | 224847_at | 0.00292 | 2.23 | CDK6 |
| 166 | 201710_at | 0.00164 | 2.21 | MYBL2 |
| 167 | 207396_s_at | 0.000756 | 2.20 | ALG3 |
| 168 | 201786_s_at | 0.00378 | 2.20 | ADAR |
| 169 | 212297_at | 0.0022 | 2.19 | KIAA0746 |
| 170 | 212644_s_at | 0.00282 | 2.19 | LHFPL2 |
| 171 | 202874_s_at | 0.00361 | 2.17 | ATP6V1C1 |
| 172 | 201462_at | 0.000757 | 2.17 | SCRN1 |
| 173 | 223003_at | 0.00421 | 2.16 | TXNDC12 |
| 174 | 201762_s_at | 0.00473 | 2.13 | PSME2 |
| 175 | 200875_s_at | 0.00376 | 2.13 | NOLSA |
| 176 | 202771_at | 0.00219 | 2.13 | FAM38A |
| 177 | 209251_x_at | 0.00243 | 2.11 | TUBA1C |
| 178 | 225435_at | 0.00298 | 2.09 | NUDCD1 |
| 179 | 203552_at | 0.00445 | 2.09 | MAP4K5 |
| 180 | 201587_s_at | 0.00302 | 2.05 | IRAK1 |
| 181 | 221058_s_at | 0.00491 | 2.02 | COTL1 |
| 182 | 202180_s_at | 0.00149 | 1.98 | MVP |
| 183 | 200959_at | 0.00344 | 1.97 | FUS |
| 184 | 200833_s_at | 0.00416 | 1.96 | hCG_1757335 /// |
| | | | | RAP1B |
| 185 | 224726_at | 0.00388 | 1.95 | WDR68 |
| 186 | 225890_at | 0.00499 | 1.94 | MARCKS |
| 187 | 222451_s_at | 0.000506 | 1.92 | LIMA1 |
| 188 | 225234_at | 0.00108 | 1.91 | FBLIM1 |
| 189 | 224777_s_at | 0.000574 | 1.88 | RBM17 |
| 190 | 203181_x_at | 0.00365 | 1.88 | SRPK2 |
| 191 | 209906_at | 0.00258 | 1.87 | C3AR1 |
| 192 | 1559822_s_at | 0.00333 | 1.83 | LOC644215 |
| 193 | 225475_at | 0.00447 | 1.82 | MFHAS1 |
| 194 | 215696_s_at | 0.00257 | 1.81 | SLC6A2 |
| 195 | 203396_at | 0.00175 | 1.80 | PSMA4 |
| 196 | 218768_at | 0.00172 | 1.75 | TMEM39B |
| 197 | 202306_at | 0.00282 | 1.73 | POLR2G |
| 198 | 213119_at | 0.00144 | 1.72 | PTPN2 |
| 199 | 221555_x_at | 0.00343 | 1.67 | MIS12 |
| 200 | 203114_at | 0.00408 | 1.63 | SSSCA1 |
| 201 | 215222_x_at | 0.00456 | 1.63 | IGL@ /// IGLJ3 |
| | | | | /// IGLV2-14 /// |
| | | | | IGLV3-25 |
| 202 | 212740_at | 0.0041 | 1.61 | NFATC2IP |
| 203 | 218089_at | 0.00069 | 1.58 | HRB |
| 204 | 226054_at | 0.00472 | 1.58 | RNF145 |
| 205 | 200096_s_at | 0.00381 | 1.58 | ATP6V0E1 |
| 206 | 203677_s_at | 0.00273 | 1.54 | TARBP2 |
| 207 | 224804_s_at | 0.0031 | −1.72 | SORT1 |
| 208 | 221527_s_at | 0.0031 | −1.93 | LSG1 |
| 209 | 211474_s_at | 0.00235 | −2.29 | BAG1 |
| 210 | 203571_s_at | 0.00247 | −2.79 | C10orf116 |
| 211 | 223183_at | 0.00416 | −2.94 | TMEM189 |
| 212 | 219298_at | 0.00438 | −5.75 | DERL1 |

TABLE III
Differentially expressed genes in the
recurrent oral tongue tumors (p < 0.05)

| Sl | | | | |
|---|---|---|---|---|
| NO | Affymetrix ID | P-value | Fold | Gene Symbol |
| 1 | 204475_at | 0.00519 | 74.50 | MMP1 |
| 2 | 205828_at | 0.0141 | 26.15 | MMP3 |
| 3 | 205680_at | 0.0151 | 23.70 | MMP10 |
| 4 | 211964_at | 0.00664 | 11.14 | COL4A2 |
| 5 | 211980_at | 0.0103 | 8.53 | COL4A1 |
| 6 | 221730_at | 0.0179 | 7.79 | COL5A2 |
| 7 | 205479_s_at | 0.00409 | 7.66 | PLAU |
| 8 | 212488_at | 0.0197 | 7.18 | COL5A1 |
| 9 | 204567_s_at | 3.83E−05 | 6.71 | ABCG1 |
| 10 | 225285_at | 0.0196 | 6.16 | BCAT1 |
| 11 | 203562_at | 0.00837 | 6.14 | FEZ1 |
| 12 | 221898_at | 0.0148 | 5.97 | PDPN |
| 13 | 210986_s_at | 0.014 | 5.86 | TPM1 |
| 14 | 209651_at | 0.0105 | 5.46 | TGFB1I1 |
| 15 | 226876_at | 0.013 | 5.45 | FAM101B |
| 16 | 203417_at | 0.00596 | 5.40 | MFAP2 |
| 17 | 203065_s_at | 0.0194 | 5.31 | CAV1 |
| 18 | 236565_s_at | 0.0145 | 5.12 | LARP6 |
| 19 | 221261_x_at | 0.0183 | 5.10 | MAGED4 /// |
| | | | | MAGED4B |
| 20 | 208091_s_at | 0.0188 | 4.85 | ECOP |
| 21 | 201185_at | 0.0118 | 4.48 | HTRA1 |
| 22 | 204992_s_at | 0.0164 | 3.94 | PFN2 |
| 23 | 230563_at | 0.0173 | 3.91 | RASGEF1A |
| 24 | 209014_at | 0.00713 | 3.89 | MAGED1 |
| 25 | 204359_at | 0.0168 | 3.81 | FLRT2 |
| 26 | 225685_at | 0.00933 | 3.77 | — |
| 27 | 202185_at | 0.0129 | 3.72 | PLOD3 |
| 28 | 211071_s_at | 0.00101 | 3.67 | MLLT11 |
| 29 | 221538_s_at | 0.00821 | 3.56 | PLXNA1 |
| 30 | 218847_at | 0.0159 | 3.56 | IGF2BP2 |
| 31 | 221641_s_at | 0.00499 | 3.37 | ACOT9 |
| 32 | 204140_at | 0.0182 | 3.33 | TPST1 |
| 33 | 224374_s_at | 0.0174 | 3.33 | EMILIN2 |
| 34 | 204924_at | 0.0118 | 3.32 | TLR2 |
| 35 | 202897_at | 0.00538 | 3.31 | SIRPA |
| 36 | 218618_s_at | 0.0165 | 3.22 | FNDC3B |
| 37 | 204589_at | 0.00788 | 3.19 | NUAK1 |
| 38 | 207714_s_at | 0.0109 | 3.18 | SERPINH1 |
| 39 | 209682_at | 0.0163 | 3.16 | CBLB |
| 40 | 225898_at | 0.00674 | 3.13 | WDR54 |
| 41 | 204030_s_at | 0.0191 | 3.11 | SCHIP1 |
| 42 | 201272_at | 0.0012 | 3.09 | AKR1B1 |
| 43 | 203823_at | 0.015 | 2.96 | RGS3 |
| 44 | 214953_s_at | 0.0198 | 2.95 | APP |
| 45 | 204083_s_at | 0.0154 | 2.91 | TPM2 |
| 46 | 219477_s_at | 0.01 | 2.89 | THSD1 /// |
| | | | | THSD1P |
| 47 | 218718_at | 0.00781 | 2.77 | PDGFC |
| 48 | 203217_s_at | 0.0172 | 2.73 | ST3GAL5 |
| 49 | 208178_x_at | 0.0181 | 2.71 | TRIO |
| 50 | 220941_s_at | 0.0179 | 2.71 | C21orf91 |
| 51 | 225303_at | 0.0185 | 2.68 | KIRREL |
| 52 | 212169_at | 0.0157 | 2.67 | FKBP9 |
| 53 | 225841_at | 0.0127 | 2.67 | C1orf59 |
| 54 | 212117_at | 0.0107 | 2.63 | RHOQ |
| 55 | 202570_s_at | 0.00666 | 2.46 | DLGAP4 |
| 56 | 202027_at | 0.00911 | 2.40 | TMEM184B |
| 57 | 204214_s_at | 0.0158 | 2.37 | RAB32 |
| 58 | 230275_at | 0.0198 | 2.29 | ARSI |
| 59 | 208079_s_at | 0.0159 | 2.23 | AURKA |
| 60 | 222622_at | 0.0191 | 2.22 | LOC283871 |
| 61 | 209784_s_at | 0.00359 | 2.21 | JAG2 |
| 62 | 203580_s_at | 0.00843 | 2.18 | SLC7A6 |
| 63 | 55093_at | 0.0191 | 2.18 | CSGlcA-T |
| 64 | 203140_at | 0.0122 | 2.18 | BCL6 |
| 65 | 227484_at | 0.00692 | 2.17 | — |
| 66 | 223095_at | 0.0186 | 2.10 | MARVELD1 |
| 67 | 205449_at | 0.0124 | 1.98 | SAC3D1 |
| 68 | 224995_at | 0.0169 | 1.96 | SPIRE1 |
| 69 | 219394_at | 0.00269 | 1.95 | PGS1 |
| 70 | 204169_at | 0.00234 | 1.95 | IMPDH1 |
| 71 | 212457_at | 0.0189 | 1.90 | TFE3 |
| 72 | 226373_at | 0.00563 | 1.86 | SFXN5 |
| 73 | 212663_at | 0.00975 | 1.85 | FKBP15 |
| 74 | 220974_x_at | 0.00877 | 1.84 | SFXN3 |
| 75 | 217855_x_at | 0.00633 | 1.78 | SDF4 |
| 76 | 212740_at | 0.0171 | 1.76 | PIK3R4 |
| 77 | 226738_at | 0.000415 | 1.74 | WDR81 |
| 78 | 219224_x_at | 0.00635 | 1.68 | ZNF408 |
| 79 | 49329_at | 0.0174 | 1.66 | KLHL22 |
| 80 | 236275_at | 0.0156 | 1.64 | KRBA1 |
| 81 | 204826_at | 0.0187 | 1.64 | CCNF |
| 82 | 38069_at | 0.0179 | 1.64 | CLCN7 |
| 83 | 217196_s_at | 0.0172 | 1.61 | CAMSAP1L1 |
| 84 | 218089_at | 0.0142 | 1.60 | C20orf4 |
| 85 | 218991_at | 0.00386 | 1.56 | HEATR6 |
| 86 | 40093_at | 0.0189 | 1.54 | BCAM |
| 87 | 211066_x_at | 0.00365 | 1.52 | PCDHGA1 /// |
| | | | | PCDHGA10 /// |
| | | | | PCDHGA11 /// |
| | | | | PCDHGA12 /// |
| | | | | PCDHGA2 /// |
| | | | | PCDHGA3 /// |
| | | | | PCDHGA4 /// |
| | | | | PCDHGA5 /// |
| | | | | PCDHGA6 /// |
| | | | | PCDHGA7 /// |
| | | | | PCDHGA8 /// |
| | | | | PCDHGA9 /// |
| | | | | PCDHGB1 /// |
| | | | | PCDHGB2 /// |
| | | | | PCDHGB3 /// |
| | | | | PCDHGB4 /// |
| | | | | PCDHGB5 /// |
| | | | | PCDHGB6 /// |
| | | | | PCDHGB7 /// |
| | | | | PCDHGC3 /// |
| | | | | PCDHGC4 /// |
| | | | | PCDHGC5 |
| 88 | 213351_s_at | 0.019 | 1.50 | TMCC1 |
| 89 | 228852_at | 0.0102 | −1.72 | ENSA |
| 90 | 223245_at | 0.0159 | −1.86 | STRBP |
| 91 | 214106_s_at | 0.0123 | −1.87 | GMDS |
| 92 | 223497_at | 0.0158 | −1.90 | FAM135A |
| 93 | 228013_at | 0.00988 | −2.01 | — |
| 94 | 230083_at | 0.00189 | −2.07 | USP53 |
| 95 | 204485_s_at | 0.0154 | −2.10 | TOM1L1 |
| 96 | 239069_s_at | 0.0149 | −2.22 | — |
| 97 | 229498_at | 0.00893 | −2.27 | — |
| 98 | 225508_at | 0.0195 | −2.75 | KIAA1468 |
| 99 | 203711_s_at | 0.0163 | −3.16 | HIBCH |
| 100 | 231270_at | 0.00951 | −3.27 | CA13 |
| 101 | 213572_s_at | 0.0165 | −3.39 | SERPINB1 |
| 102 | 213050_at | 0.0114 | −3.76 | COBL |
| 103 | 221523_s_at | 0.0102 | −4.00 | RRAGD |
| 104 | 223822_at | 0.00826 | −4.18 | SUSD4 |
| 105 | 213895_at | 0.0158 | −4.50 | EMP1 |
| 106 | 218858_at | 0.0199 | −4.59 | DEPDC6 |
| 107 | 231929_at | 0.00177 | −6.58 | IKZF2 |
| 108 | 214063_s_at | 0.0116 | −6.67 | TF |
| 109 | 231145_at | 0.0184 | −7.19 | — |
| 110 | 209498_at | 0.0174 | −7.69 | CEACAM1 |
| 111 | 1559606_at | 0.0192 | −11.51 | GBP6 |
| 112 | 220026_at | 0.00299 | −16.26 | CLCA4 |

TABLE IV
Nonrecurrent Tumor versus Recurrent Tumor

| Sl | | | Fold | |
|---|---|---|---|---|
| NO | Affymetrix ID | p-value | (NR/R) | Gene Symbol |
| 1 | 220690_s_at | 6.62E−06 | 1.83 | DHRS7B |
| 2 | 208614_s_at | 0.000933 | −2.04 | FLNB |
| 3 | 226012_at | 0.00115 | −1.80 | ANKRD11 |
| 4 | 222768_s_at | 0.00217 | 1.76 | TRMT6 |
| 5 | 211959_at | 0.00223 | 5.81 | IGFBP5 |
| 6 | 242989_at | 0.0034 | −1.53 | — |
| 7 | 218281_at | 0.00363 | 1.70 | MRPL48 |
| 8 | 223413_s_at | 0.00389 | 1.92 | LYAR |
| 9 | 201582_at | 0.00414 | 1.58 | SEC23B |
| 10 | 200805_at | 0.00421 | 1.77 | LMAN2 |
| 11 | 222437_s_at | 0.00608 | 1.72 | VPS24 |
| 12 | 218235_s_at | 0.00647 | 1.64 | UTP11L |
| 13 | 218841_at | 0.00659 | 1.66 | ASB8 |
| 14 | 203424_s_at | 0.0077 | 2.47 | IGFBP5 |
| 15 | 218225_at | 0.00791 | 1.52 | ECSIT |
| 16 | 209054_s_at | 0.00797 | −1.51 | WHSC1 |
| 17 | 226426_at | 0.00811 | −1.58 | — |
| 18 | 225192_at | 0.00834 | −1.79 | C10orf46 |
| 19 | 226605_at | 0.00888 | −1.52 | DGKQ |
| 20 | 209283_at | 0.00949 | 3.76 | CRYAB |
| 21 | 220201_at | 0.00996 | −1.83 | RC3H2 |
| 22 | 217973_at | 0.0102 | 2.31 | DCXR |
| 23 | 213189_at | 0.0102 | 1.79 | MINA |
| 24 | 202471_s_at | 0.0103 | 1.60 | IDH3G |
| 25 | 208906_at | 0.0124 | 1.72 | BSCL2 /// HNRPUL2 |
| 26 | 201052_s_at | 0.0128 | 1.68 | PSMF1 |
| 27 | 208675_s_at | 0.0138 | 1.68 | DDOST |
| 28 | 204868_at | 0.0139 | 1.93 | ICT1 |
| 29 | 209355_s_at | 0.014 | 3.68 | PPAP2B |
| 30 | 208003_s_at | 0.014 | −2.44 | NFAT5 |
| 31 | 202357_s_at | 0.0146 | 2.28 | CFB |
| 32 | 228159_at | 0.015 | −1.74 | — |
| 33 | 202433_at | 0.0154 | 1.58 | SLC35B1 |
| 34 | 210125_s_at | 0.016 | 2.32 | BANF1 |
| 35 | 218462_at | 0.0163 | 1.56 | BXDC5 |
| 36 | 212135_s_at | 0.0167 | −1.56 | ATP2B4 |
| 37 | 200917_s_at | 0.017 | 2.30 | SRPR |
| 38 | 200846_s_at | 0.0173 | 1.79 | PPP1CA |
| 39 | 221667_s_at | 0.0174 | 2.49 | HSPB8 |
| 40 | 201583_s_at | 0.0175 | 1.89 | SEC23B |
| 41 | 209575_at | 0.0177 | 1.87 | IL10RB |
| 42 | 209742_s_at | 0.0179 | 6.82 | MYL2 |
| 43 | 225868_at | 0.0184 | 1.60 | TRIM47 |
| 44 | 217884_at | 0.0194 | −1.59 | NAT10 |
| 45 | 208800_at | 0.0203 | 1.51 | SRP72 |
| 46 | 219348_at | 0.0206 | 1.68 | USE1 |
| 47 | 208238_x_at | 0.0208 | −1.55 | — |
| 48 | 212411_at | 0.0212 | 1.62 | IMP4 |
| 49 | 219217_at | 0.023 | 1.52 | NARS2 |
| 50 | 202412_s_at | 0.0236 | 1.90 | USP1 |
| 51 | 226043_at | 0.0246 | −1.83 | GPSM1 |
| 52 | 228310_at | 0.0249 | −1.92 | ENAH |
| 53 | 203391_at | 0.0261 | 1.66 | FKBP2 |
| 54 | 233814_at | 0.0265 | 2.26 | — |
| 55 | 203734_at | 0.0265 | −1.69 | FOXJ2 |
| 56 | 203022_at | 0.0276 | 1.87 | RNASEH2A |
| 57 | 209030_s_at | 0.0277 | 2.02 | CADM1 |
| 58 | 208991_at | 0.0283 | 1.53 | STAT3 |
| 59 | 213523_at | 0.0285 | −1.83 | CCNE1 |
| 60 | 216032_s_at | 0.029 | 1.52 | ERGIC3 |
| 61 | 227547_at | 0.0291 | −1.56 | — |
| 62 | 207621_s_at | 0.0293 | 1.77 | PEMT |
| 63 | 204839_at | 0.0295 | 1.78 | POP5 |
| 64 | 223203_at | 0.0298 | −1.58 | TMEM29 /// TMEM29B |
| 65 | 202905_x_at | 0.0299 | 1.65 | NBN |
| 66 | 1553709_a_at | 0.0299 | 1.63 | PRPF38A |
| 67 | 204074_s_at | 0.0302 | 1.53 | KIAA0562 |
| 68 | 224646_x_at | 0.0311 | 5.66 | H19 |
| 69 | 201145_at | 0.0311 | 1.70 | HAX1 |
| 70 | 201532_at | 0.0321 | 1.58 | PSMA3 |
| 71 | 212861_at | 0.0328 | 1.82 | MFSD5 |
| 72 | 218400_at | 0.0334 | 2.76 | OAS3 |
| 73 | 224609_at | 0.0337 | 1.93 | SLC44A2 |
| 74 | 208887_at | 0.0338 | 1.56 | EIF3G |
| 75 | 1553551_s_at | 0.0343 | 1.95 | — |
| 76 | 218258_at | 0.0345 | 1.54 | POLR1D |
| 77 | 228123_s_at | 0.035 | 1.86 | ABHD12 |
| 78 | 223210_at | 0.0351 | 2.29 | CHURC1 |
| 79 | 221188_s_at | 0.0352 | 1.59 | CIDEB |
| 80 | 237563_s_at | 0.0361 | 3.01 | LOC440731 |
| 81 | 1555653_at | 0.0362 | 2.01 | HNRPA3 |
| 82 | 229322_at | 0.0362 | 1.58 | PPP2R5E |
| 83 | 202109_at | 0.0366 | 1.63 | ARFIP2 |
| 84 | 203872_at | 0.0368 | 9.79 | ACTA1 |
| 85 | 203082_at | 0.0371 | −1.59 | BMS1 |
| 86 | 201659_s_at | 0.0373 | 1.73 | ARL1 |
| 87 | 211745_x_at | 0.0376 | 6.52 | HBA1 |
| 88 | 211600_at | 0.0376 | 2.17 | — |
| 89 | 209458_x_at | 0.0378 | 5.23 | HBA1 /// HBA2 |
| 90 | 213201_s_at | 0.0378 | 2.51 | TNNT1 |
| 91 | 227864_s_at | 0.0384 | 2.00 | FAM125A |
| 92 | 222527_s_at | 0.0385 | 1.79 | RBM22 |
| 93 | 209904_at | 0.0386 | 5.12 | TNNC1 |
| 94 | 228261_at | 0.0386 | 2.43 | MIB2 |
| 95 | 201534_s_at | 0.0387 | 1.92 | UBL3 |
| 96 | 212922_s_at | 0.039 | 1.58 | SMYD2 |
| 97 | 243720_at | 0.0395 | −1.91 | CMIP |
| 98 | 235674_at | 0.0396 | −1.52 | KIAA0922 |
| 99 | 227276_at | 0.0399 | 1.98 | PLXDC2 |
| 100 | 225058_at | 0.0399 | 1.56 | GPR108 |
| 101 | 228408_s_at | 0.04 | 1.70 | SDAD1 |
| 102 | 203090_at | 0.0401 | 1.51 | SDF2 |
| 103 | 208717_at | 0.0404 | 1.53 | OXA1L |
| 104 | 221998_s_at | 0.0405 | 1.89 | VRK3 |
| 105 | 221486_at | 0.0406 | 1.62 | ENSA |
| 106 | 201264_at | 0.0408 | 2.64 | COPE |
| 107 | 202036_s_at | 0.0409 | 3.43 | SFRP1 |
| 108 | 209852_x_at | 0.0412 | 1.83 | PSME3 |
| 109 | 242844_at | 0.0413 | 1.69 | PGGT1B |
| 110 | 226316_at | 0.0416 | −1.87 | — |
| 111 | 211699_x_at | 0.0422 | 4.50 | HBA1 /// HBA2 |
| 112 | 205374_at | 0.0427 | 6.84 | SLN |
| 113 | 203882_at | 0.0428 | 2.10 | IRF9 |
| 114 | 212654_at | 0.043 | 3.51 | TPM2 |
| 115 | 208705_s_at | 0.043 | 1.98 | EIF5 |
| 116 | 219428_s_at | 0.0431 | 1.66 | PXMP4 |
| 117 | 204018_x_at | 0.0441 | 4.61 | HBA1 /// HBA2 |
| 118 | 228843_at | 0.0443 | −2.03 | — |
| 119 | 222233_s_at | 0.0447 | 1.91 | DCLRE1C |
| 120 | 220952_s_at | 0.0453 | −1.65 | PLEKHA5 |
| 121 | 219772_s_at | 0.0458 | 4.95 | SMPX |
| 122 | 209116_x_at | 0.0462 | 11.92 | HBB |
| 123 | 228222_at | 0.0463 | 2.07 | PPP1CB |
| 124 | 204179_at | 0.0466 | 9.45 | MB |
| 125 | 204810_s_at | 0.047 | 7.64 | CKM |
| 126 | 200820_at | 0.0473 | 1.65 | PSMD8 |
| 127 | 202296_s_at | 0.0474 | 1.63 | RER1 |
| 128 | 208627_s_at | 0.0476 | 1.65 | YBX1 |
| 129 | 201161_s_at | 0.0477 | 1.52 | CSDA |
| 130 | 225294_s_at | 0.0478 | 1.92 | TRAPPC1 |
| 131 | 225978_at | 0.0482 | −1.85 | FAM80B |
| 132 | 217192_s_at | 0.0487 | 2.09 | PRDM1 |
| 133 | 217232_x_at | 0.0489 | 8.07 | HBB |
| 134 | 202037_s_at | 0.0491 | 4.64 | SFRP1 |
| 135 | 239057_at | 0.0492 | 2.87 | LMOD2 |
| 136 | 214141_x_at | 0.0493 | 1.60 | SFRS7 |
| 137 | 201263_at | 0.0494 | 1.65 | TARS |
| 138 | 209888_s_at | 0.0497 | 6.38 | MYL1 |
| 139 | 214102_at | 0.0497 | −1.63 | CENTD1 |
| 140 | 220248_x_at | 0.0499 | 1.59 | NSFL1C |

TABLE V
Normal: NonRecurrent versus Normal Recurrent (adjacent mucosa)

| Sl | | | Fold | |
|---|---|---|---|---|
| No | Affymetrix ID | P-value | (NR/R) | Gene Symbol |
| 1 | 238035_at | 0.00212 | −1.96 | SP3 |
| 2 | 217232_x_at | 0.00256 | 23.81 | HBB |
| 3 | 225633_at | 0.00267 | −2.30 | DPY19L3 |
| 4 | 209116_x_at | 0.00287 | 33.93 | HBB |
| 5 | 211696_x_at | 0.00288 | 21.34 | HBB |
| 6 | 228238_at | 0.00379 | −4.15 | GAS5 |
| 7 | 225997_at | 0.00491 | −1.80 | MOBKL1A |
| 8 | 237646_x_at | 0.00492 | 1.76 | PLEKHG5 |
| 9 | 210873_x_at | 0.00523 | −20.12 | APOBEC3A |
| 10 | 209405_s_at | 0.00539 | 1.99 | FAM3A |
| 11 | 34689_at | 0.00569 | 1.94 | TREX1 |
| 12 | 223415_at | 0.00617 | 1.72 | RPP25 |
| 13 | 212476_at | 0.00619 | −1.99 | CENTB2 |
| 14 | 200069_at | 0.0072 | −1.83 | SART3 |
| 15 | 205236_x_at | 0.00742 | 1.94 | SOD3 |
| 16 | 205784_x_at | 0.00748 | 1.88 | ARVCF |
| 17 | 212134_at | 0.00771 | 2.07 | PHLDB1 |
| 18 | 238066_at | 0.00798 | 2.57 | RBP7 |
| 19 | 203045_at | 0.00848 | 2.93 | NINJ1 |
| 20 | 211967_at | 0.00856 | −2.67 | TMEM123 |
| 21 | 242039_at | 0.00881 | 1.78 | CENTD2 |
| 22 | 217040_x_at | 0.00915 | 1.97 | SOX15 |
| 23 | 212474_at | 0.00981 | −2.37 | KIAA0241 |
| 24 | 209420_s_at | 0.00983 | 1.94 | SMPD1 |
| 25 | 224726_at | 0.0101 | −1.79 | MIB1 |
| 26 | 212782_x_at | 0.0102 | 3.03 | POLR2J |
| 27 | 212910_at | 0.0103 | 1.94 | THAP11 |
| 28 | 213111_at | 0.0106 | −1.67 | PIP5K3 |
| 29 | 242989_at | 0.0108 | −2.79 | — |
| 30 | 209849_s_at | 0.0112 | 1.91 | RAD51C |
| 31 | 226109_at | 0.0112 | −2.27 | C21orf91 |
| 32 | 1557521_a_at | 0.0116 | −4.12 | — |
| 33 | 225433_at | 0.0118 | −1.69 | GTF2A1 |
| 34 | 228980_at | 0.0119 | −2.41 | RFFL |
| 35 | 212064_x_at | 0.0122 | 1.63 | MAZ |
| 36 | 218050_at | 0.0123 | −2.10 | UFM1 |
| 37 | 211745_x_at | 0.0124 | 22.72 | HBA1 |
| 38 | 221274_s_at | 0.0124 | 1.76 | LMAN2L |
| 39 | 201928_at | 0.0124 | −1.76 | PKP4 |
| 40 | 203552_at | 0.0124 | −2.40 | MAP4K5 |
| 41 | 230046_at | 0.0125 | 1.63 | — |
| 42 | 212900_at | 0.0125 | −2.17 | SEC24A |
| 43 | 215778_x_at | 0.0126 | 1.96 | HAB1 |
| 44 | 209398_at | 0.0129 | 3.89 | HIST1H1C |
| 45 | 209798_at | 0.0131 | −1.89 | NPAT |
| 46 | 218896_s_at | 0.0132 | −2.49 | C17orf85 |
| 47 | 225479_at | 0.0136 | −1.90 | LRRC58 |
| 48 | 212037_at | 0.0136 | −2.02 | PNN |
| 49 | 238563_at | 0.0136 | −3.91 | — |
| 50 | 222627_at | 0.0144 | −1.83 | VPS54 |
| 51 | 227679_at | 0.0146 | 1.97 | — |
| 52 | 203569_s_at | 0.0147 | −1.67 | OFD1 |
| 53 | 201088_at | 0.015 | −3.09 | KPNA2 |
| 54 | 212771_at | 0.0151 | 1.70 | C10orf38 |
| 55 | 238326_at | 0.0152 | 1.97 | LOC440836 |
| 56 | 212705_x_at | 0.0153 | 2.13 | PNPLA2 |
| 57 | 201468_s_at | 0.0156 | −2.34 | NQO1 |
| 58 | 202933_s_at | 0.0162 | −2.21 | YES1 |
| 59 | 211240_x_at | 0.0162 | −2.53 | CTNND1 |
| 60 | 228487_s_at | 0.0163 | −1.57 | — |
| 61 | 218750_at | 0.0163 | −3.79 | JOSD3 |
| 62 | 217414_x_at | 0.0166 | 15.81 | HBA1 /// HBA2 |
| 63 | 221600_s_at | 0.0166 | 2.00 | C11orf67 |
| 64 | 223141_at | 0.0166 | 1.57 | UCK1 |
| 65 | 208809_s_at | 0.0168 | −2.72 | C6orf62 |
| 66 | 225318_at | 0.0169 | −1.98 | — |
| 67 | 218330_s_at | 0.0169 | −2.15 | NAV2 |
| 68 | 203421_at | 0.017 | 1.89 | TP53I11 |
| 69 | 234918_at | 0.0171 | 1.66 | GLTSCR2 |
| 70 | 226217_at | 0.0171 | −2.45 | SLC30A7 |
| 71 | 238402_s_at | 0.0172 | 1.79 | FLJ35220 |
| 72 | 214414_x_at | 0.0173 | 12.63 | HBA2 |
| 73 | 216180_s_at | 0.0174 | 1.66 | SYNJ2 |
| 74 | 202210_x_at | 0.0174 | 1.62 | GSK3A |
| 75 | 201845_s_at | 0.0174 | −1.91 | RYBP |
| 76 | 225310_at | 0.0174 | −2.48 | RBMX |
| 77 | 203055_s_at | 0.0176 | 2.07 | ARHGEF1 |
| 78 | 203044_at | 0.0176 | −1.72 | CHSY1 |
| 79 | 225428_s_at | 0.0179 | 1.64 | DDX54 |
| 80 | 226208_at | 0.018 | −3.64 | ZSWIM6 |
| 81 | 212047_s_at | 0.0181 | 1.79 | RNF167 |
| 82 | 208918_s_at | 0.0182 | −1.79 | NADK |
| 83 | 1566140_at | 0.0182 | −4.67 | HOPX |
| 84 | 209458_x_at | 0.0183 | 23.19 | HBA1 /// HBA2 |
| 85 | 209903_s_at | 0.0185 | −1.99 | ATR |
| 86 | 226302_at | 0.0186 | −3.13 | ATP8B1 |
| 87 | 233849_s_at | 0.0186 | −3.15 | ARHGAP5 |
| 88 | 201458_s_at | 0.019 | −2.16 | BUB3 |
| 89 | 217696_at | 0.0192 | 1.70 | FUT7 |
| 90 | 217986_s_at | 0.0193 | −3.48 | BAZ1A |
| 91 | 228603_at | 0.0194 | −2.30 | — |
| 92 | 237046_x_at | 0.0195 | 1.66 | C16orf77 |
| 93 | 208798_x_at | 0.0199 | −2.94 | GOLGA8A |
| 94 | 225343_at | 0.0201 | −1.84 | TMED8 |
| 95 | 227642_at | 0.0202 | −3.28 | TFCP2L1 |
| 96 | 203342_at | 0.0204 | 2.01 | TIMM17B |
| 97 | 203693_s_at | 0.0204 | −3.00 | E2F3 |
| 98 | 223405_at | 0.0208 | −2.17 | NPL |
| 99 | 224935_at | 0.021 | −1.94 | EIF2S3 |
| 100 | 225731_at | 0.0212 | −2.57 | ANKRD50 |
| 101 | 225912_at | 0.0216 | −2.05 | TP53INP1 |
| 102 | 202883_s_at | 0.0216 | −2.87 | PPP2R1B |
| 103 | 200698_at | 0.0217 | −2.63 | KDELR2 |
| 104 | 222603_at | 0.0219 | −2.68 | ERMP1 |
| 105 | 203083_at | 0.0219 | −3.42 | THBS2 |
| 106 | 217776_at | 0.0221 | −2.03 | RDH11 |
| 107 | 212307_s_at | 0.0222 | −3.07 | OGT |
| 108 | 225773_at | 0.0224 | −1.96 | RSPRY1 |
| 109 | 230097_at | 0.0224 | −3.45 | GART |
| 110 | 209739_s_at | 0.0226 | 1.62 | PNPLA4 |
| 111 | 204018_x_at | 0.0232 | 16.41 | HBA1 /// HBA2 |
| 112 | 225447_at | 0.0233 | −2.04 | GPD2 |
| 113 | 225761_at | 0.0233 | −2.07 | PAPD4 |
| 114 | 212031_at | 0.0236 | −2.83 | RBM25 |
| 115 | 1556006_s_at | 0.0236 | −5.59 | CSNK1A1 |
| 116 | 232706_s_at | 0.0237 | 1.58 | TRABD |
| 117 | 200729_s_at | 0.0237 | −4.17 | ACTR2 |
| 118 | 218762_at | 0.0238 | 1.68 | ZNF574 |
| 119 | 227415_at | 0.0239 | −2.34 | LOC283508 |
| 120 | 208785_s_at | 0.024 | 2.01 | MAP1LC3B |
| 121 | 212377_s_at | 0.0242 | −1.88 | NOTCH2 |
| 122 | 227517_s_at | 0.0244 | −5.29 | GAS5 /// |
| | | | | SNORD79 |
| 123 | 202951_at | 0.0245 | −2.21 | STK38 |
| 124 | 209135_at | 0.0249 | −2.67 | ASPH |
| 125 | 218423_x_at | 0.0251 | −1.90 | VPS54 |
| 126 | 222543_at | 0.0251 | −1.98 | DERL1 |
| 127 | 227038_at | 0.0252 | −4.35 | SGMS2 |
| 128 | 208862_s_at | 0.0253 | −2.78 | CTNND1 |
| 129 | 224464_s_at | 0.0255 | 2.57 | NUDT22 |
| 130 | 210249_s_at | 0.0256 | 2.04 | NCOA1 |
| 131 | 212267_at | 0.0257 | −1.78 | WAPAL |
| 132 | 229874_x_at | 0.026 | 1.93 | LOC729604 |
| 133 | 212663_at | 0.0261 | 1.54 | FKBP15 |
| 134 | 215460_x_at | 0.0261 | −1.97 | BRD1 |
| 135 | 202200_s_at | 0.0265 | −2.50 | SRPK1 |
| 136 | 223092_at | 0.0267 | 2.36 | ANKH |
| 137 | 221503_s_at | 0.0267 | 1.74 | KPNA3 |
| 138 | 227366_at | 0.0268 | 2.30 | RILP |
| 139 | 200947_s_at | 0.0268 | −2.56 | GLUD1 |
| 140 | 227861_at | 0.027 | −1.59 | TMEM161B |
| 141 | 241650_x_at | 0.0272 | 1.56 | HMCN2 |
| 142 | 202633_at | 0.0274 | −1.79 | TOPBP1 |
| 143 | 209107_x_at | 0.0276 | 2.12 | NCOA1 |
| 144 | 203743_s_at | 0.0276 | −3.85 | TDG |
| 145 | 218247_s_at | 0.0278 | −2.34 | MEX3C |
| 146 | 218255_s_at | 0.0282 | 1.70 | FBRS |
| 147 | 225188_at | 0.0282 | −2.26 | RAPH1 |
| 148 | 211699_x_at | 0.0284 | 18.70 | HBA1 /// HBA2 |
| 149 | 224903_at | 0.0284 | −1.76 | CIRH1A |
| 150 | 229758_at | 0.0289 | 1.65 | TIGD5 |
| 151 | 212834_at | 0.029 | −2.20 | DDX52 |
| 152 | 240452_at | 0.029 | −4.26 | GSPT1 |
| 153 | 214333_x_at | 0.0293 | 1.87 | IDH3G |
| 154 | 221069_s_at | 0.0295 | 1.62 | CCDC44 |
| 155 | 218657_at | 0.0295 | −2.72 | RAPGEFL1 |
| 156 | 210613_s_at | 0.0296 | 1.71 | SYNGR1 |
| 157 | 217516_x_at | 0.03 | 1.71 | ARVCF |
| 158 | 211074_at | 0.0301 | 4.71 | FOLR1 |
| 159 | 217691_x_at | 0.0302 | 1.83 | SLC16A3 |
| 160 | 201437_s_at | 0.0302 | −1.99 | EIF4E |
| 161 | 203842_s_at | 0.0305 | 1.69 | MAPRE3 |
| 162 | 200626_s_at | 0.0305 | −1.61 | MATR3 |
| 163 | 224998_at | 0.0306 | −2.46 | CMTM4 |
| 164 | 207483_s_at | 0.0307 | −1.77 | CAND1 |
| 165 | 221840_at | 0.031 | −3.76 | PTPRE |
| 166 | 235457_at | 0.0314 | −2.29 | MAML2 |
| 167 | 227110_at | 0.0319 | −1.93 | HNRNPC |
| 168 | 224974_at | 0.032 | −2.48 | SUDS3 |
| 169 | 201916_s_at | 0.0321 | −1.97 | SEC63 |
| 170 | 218738_s_at | 0.0321 | −1.98 | RNF138 |
| 171 | 210371_s_at | 0.0321 | −2.24 | RBBP4 |
| 172 | 218940_at | 0.0323 | −2.09 | C14orf138 |
| 173 | AFFX-r2-Bs- | 0.0325 | 6.16 | — |
| | lys-3_at | | | |
| 174 | 219037_at | 0.0325 | −2.58 | RRP15 |
| 175 | 204829_s_at | 0.0328 | 2.31 | FOLR2 |
| 176 | 224467_s_at | 0.0328 | 1.96 | PDCD2L |
| 177 | 200599_s_at | 0.0329 | −1.89 | HSP90B1 |
| 178 | 225480_at | 0.0332 | 1.71 | C1orf122 |
| 179 | 227765_at | 0.0332 | 1.60 | — |
| 180 | 233011_at | 0.0332 | −18.73 | ANXA1 |
| 181 | 226965_at | 0.0334 | −2.11 | FAM116A |
| 182 | 233955_x_at | 0.0339 | 2.41 | CXXC5 |
| 183 | 1553979_at | 0.034 | −2.00 | — |
| 184 | 217879_at | 0.0342 | −1.61 | CDC27 |
| 185 | 225416_at | 0.0342 | −2.08 | RNF12 |
| 186 | 208101_s_at | 0.0343 | 1.66 | URM1 |
| 187 | 209217_s_at | 0.035 | 2.04 | WDR45 |
| 188 | 201197_at | 0.0351 | −4.27 | AMD1 |
| 189 | 220417_s_at | 0.0352 | 1.94 | LOC728944 /// |
| | | | | THAP4 |
| 190 | 218104_at | 0.0352 | −1.97 | TEX 10 |
| 191 | 212484_at | 0.0353 | 2.61 | FAM89B |
| 192 | 222742_s_at | 0.0353 | 2.42 | RABL5 |
| 193 | 89476_r_at | 0.0353 | 1.60 | NPEPL1 |
| 194 | 202009_at | 0.0357 | 2.11 | TWF2 |
| 195 | 216862_s_at | 0.0358 | 2.28 | MTCP1 |
| 196 | 203080_s_at | 0.0358 | −1.63 | BAZ2B |
| 197 | 203905_at | 0.0358 | −2.15 | PARN |
| 198 | 219983_at | 0.0359 | 4.53 | HRASLS |
| 199 | 218515_at | 0.0359 | −1.82 | C21orf66 |
| 200 | 233656_s_at | 0.0359 | −2.11 | VPS54 |
| 201 | 211692_s_at | 0.0361 | 1.62 | BBC3 |
| 202 | 226604_at | 0.0362 | −2.21 | TMTC3 |
| 203 | 209332_s_at | 0.0363 | −1.59 | MAX |
| 204 | 201456_s_at | 0.0363 | −1.77 | BUB3 |
| 205 | 225415_at | 0.0363 | −1.77 | DTX3L |
| 206 | 241799_x_at | 0.0366 | 1.57 | — |
| 207 | 209476_at | 0.0369 | −2.27 | TXNDC1 |
| 208 | 212628_at | 0.037 | −2.13 | PKN2 |
| 209 | 210212_x_at | 0.0373 | 2.13 | MTCP1 |
| 210 | 203567_s_at | 0.0375 | −1.71 | TRIM38 |
| 211 | 225284_at | 0.0376 | −1.71 | LOC144871 |
| 212 | 208152_s_at | 0.0376 | −2.46 | DDX21 |
| 213 | 213168_at | 0.0378 | −1.66 | SP3 |
| 214 | 218230_at | 0.0379 | −2.31 | ARFIP1 |
| 215 | 218595_s_at | 0.0379 | −2.62 | HEATR1 |
| 216 | 228222_at | 0.0382 | 3.04 | PPP1CB |
| 217 | 202396_at | 0.0382 | −2.48 | TCERG1 |
| 218 | 220973_s_at | 0.0383 | 2.10 | SHARPIN |
| 219 | 218743_at | 0.0383 | 1.82 | CHMP6 |
| 220 | 227586_at | 0.0385 | −1.95 | TMEM170 |
| 221 | 224959_at | 0.0385 | −3.02 | SLC26A2 |
| 222 | 218956_s_at | 0.0386 | 1.70 | PTCD1 |
| 223 | 203575_at | 0.0386 | 1.68 | CSNK2A2 |
| 224 | 226200_at | 0.0386 | 1.65 | VARS2 |
| 225 | 202603_at | 0.0386 | −1.85 | — |
| 226 | 221751_at | 0.0386 | −1.95 | SLC2A3P1 |
| 227 | 223297_at | 0.0387 | −2.34 | AMMECR1L |
| 228 | 240038_at | 0.0389 | −5.46 | — |
| 229 | 222996_s_at | 0.0391 | 2.72 | CXXC5 |
| 230 | 239392_s_at | 0.0392 | −2.49 | — |
| 231 | 202688_at | 0.0393 | 2.41 | TNFSF10 |
| 232 | 209034_at | 0.0393 | 2.02 | PNRC1 |
| 233 | 226146_at | 0.0393 | 1.81 | — |
| 234 | 225107_at | 0.0393 | −3.04 | HNRNPA2B1 |
| 235 | 202948_at | 0.0395 | −1.54 | IL1R1 |
| 236 | 204300_at | 0.0396 | 2.01 | PET112L |
| 237 | 212066_s_at | 0.0396 | −1.60 | USP34 |
| 238 | 209666_s_at | 0.0396 | −2.00 | CHUK |
| 239 | 208003_s_at | 0.0397 | −2.35 | NFAT5 |
| 240 | AFFX-PheX- | 0.0399 | 4.60 | — |
| | 3_at | | | |
| 241 | 221918_at | 0.0399 | −1.51 | PCTK2 |
| 242 | 218803_at | 0.0399 | −2.30 | CHFR |
| 243 | 225973_at | 0.0399 | −3.51 | TAP2 |
| 244 | 218533_s_at | 0.0402 | 3.47 | UCKL1 |
| 245 | 200783_s_at | 0.0402 | −2.07 | STMN1 |
| 246 | 231513_at | 0.0404 | 5.16 | — |
| 247 | 221802_s_at | 0.0405 | −4.95 | KIAA1598 |
| 248 | 203775_at | 0.0406 | −3.10 | SLC25A13 |
| 249 | 227878_s_at | 0.0407 | 2.38 | ALKBH7 |
| 250 | 202135_s_at | 0.0407 | 1.88 | ACTR1B |
| 251 | 201795_at | 0.0407 | −1.93 | LBR |
| 252 | 212293_at | 0.0408 | −1.98 | HIPK1 |
| 253 | 212378_at | 0.0408 | −2.42 | GART |
| 254 | 212228_s_at | 0.041 | 4.02 | COQ9 |
| 255 | 203719_at | 0.041 | 2.09 | ERCC1 |
| 256 | 225361_x_at | 0.0412 | −1.87 | FAM122B |
| 257 | 225643_at | 0.0413 | −2.21 | C14orf32 |
| 258 | 223497_at | 0.0413 | −2.74 | FAM135A |
| 259 | 212033_at | 0.0418 | −2.03 | RBM25 |
| 260 | 212721_at | 0.042 | −1.90 | SFRS12 |
| 261 | 220734_s_at | 0.0421 | 2.21 | GLTPD1 /// |
| | | | | LOC727825 |
| 262 | 206453_s_at | 0.0422 | 2.52 | NDRG2 |
| 263 | 201704_at | 0.0423 | −1.52 | ENTPD6 |
| 264 | 1554480_a_at | 0.0426 | −1.56 | ARMC10 |
| 265 | 223398_at | 0.0427 | 1.75 | C9orf89 |
| 266 | 228677_s_at | 0.0428 | 1.86 | FLJ21438 |
| 267 | 224887_at | 0.0428 | 1.55 | GNPTG |
| 268 | 215696_s_at | 0.0428 | −1.99 | SEC16A |
| 269 | 202778_s_at | 0.043 | −1.91 | ZMYM2 |
| 270 | 224866_at | 0.0431 | −4.00 | MLSTD2 |
| 271 | 1553955_at | 0.0432 | −2.16 | CCDC128 |
| 272 | 213056_at | 0.0433 | −4.44 | FRMD4B |
| 273 | 224436_s_at | 0.0435 | −1.75 | NIPSNAP3A |
| 274 | 225785_at | 0.0435 | −1.83 | REEP3 |
| 275 | 201873_s_at | 0.0437 | −2.16 | ABCE1 |
| 276 | 208907_s_at | 0.0439 | 2.28 | MRPS18B |
| 277 | 224415_s_at | 0.044 | 2.59 | HINT2 |
| 278 | 223281_s_at | 0.0443 | 1.69 | COX15 |
| 279 | 218647_s_at | 0.0443 | −2.80 | YRDC |
| 280 | 218499_at | 0.0443 | −5.59 | RP6-213H19.1 |
| 281 | 225534_at | 0.0445 | 2.62 | C8orf40 |
| 282 | 212163_at | 0.0445 | −1.72 | KIDINS220 |
| 283 | 204469_at | 0.0445 | −10.31 | PTPRZ1 |
| 284 | 201586_s_at | 0.0446 | −3.00 | SFPQ |
| 285 | 218227_at | 0.0447 | 1.67 | NUBP2 |
| 286 | 221903_s_at | 0.0447 | −2.26 | CYLD |
| 287 | 233571_x_at | 0.0449 | 1.94 | C20orf149 |
| 288 | 212160_at | 0.0449 | −2.09 | XPOT |
| 289 | 219922_s_at | 0.045 | 2.17 | LTBP3 |
| 290 | 202996_at | 0.0451 | 1.55 | POLD4 |
| 291 | 223072_s_at | 0.0452 | 1.65 | WBP1 |
| 292 | 201091_s_at | 0.0452 | −1.81 | CBX3 /// |
| | | | | LOC653972 |
| 293 | 227624_at | 0.0453 | −2.35 | KIAA1546 |
| 294 | 226538_at | 0.0457 | −1.53 | MAN2A1 |
| 295 | 220934_s_at | 0.0459 | 2.16 | MGC3196 |
| 296 | 228135_at | 0.0459 | −1.59 | C1orf52 |
| 297 | 227422_at | 0.046 | −2.17 | — |
| 298 | 218984_at | 0.0461 | −2.15 | PUS7 |
| 299 | 226003_at | 0.0463 | −4.05 | KIF21A |
| 300 | 229009_at | 0.0466 | 1.96 | SIX5 |
| 301 | 1554149_at | 0.0469 | −1.75 | CLDND1 |
| 302 | 223050_s_at | 0.0471 | 2.34 | FBXW5 |
| 303 | 202314_at | 0.0471 | −3.31 | CYP51A1 |
| 304 | 212533_at | 0.0471 | −4.31 | WEE1 |
| 305 | 221163_s_at | 0.0475 | 2.36 | MLXIPL |
| 306 | 205968_at | 0.0477 | 2.44 | KCNS3 |
| 307 | 200055_at | 0.0477 | 1.82 | TAF10 |
| 308 | 218841_at | 0.048 | 3.72 | ASB8 |
| 309 | 202399_s_at | 0.048 | 1.62 | AP3S2 |
| 310 | 203020_at | 0.0482 | −1.81 | RABGAP1L |
| 311 | 222673_x_at | 0.0483 | −1.88 | FAM122B /// |
| | | | | TMEM57 |
| 312 | 201939_at | 0.0483 | −3.32 | PLK2 |
| 313 | 205436_s_at | 0.0484 | 1.78 | H2AFX |
| 314 | 204565_at | 0.0486 | 2.97 | THEM2 |
| 315 | 211368_s_at | 0.0486 | −2.79 | CASP1 |
| 316 | 223454_at | 0.0486 | −2.95 | CXCL16 |
| 317 | 223312_at | 0.0487 | 2.72 | C2orf7 |
| 318 | 214213_x_at | 0.0488 | 1.54 | LMNA |
| 319 | 202799_at | 0.0489 | 2.14 | CLPP |
| 320 | 203739_at | 0.0493 | −3.72 | ZNF217 |
| 321 | 220952_s_at | 0.0495 | −2.29 | PLEKHA5 |
| 322 | 203358_s_at | 0.0498 | −4.76 | EZH2 |
| 323 | 212540_at | 0.05 | 1.99 | CDC34 |

TABLE VI
Clinical Characteristics of patients

| | | | | | Med | |
|---|---|---|---|---|---|---|
| | | Med | Risk habits# | | Follow | Med |
| | Sample | Age | With | Without | up | DFS |
| Study | size | (Years) | Risk | Risk | (months) | (months) |
| Microarray | 12 | 54.5 | 6 | 6 | 47 | |
| Set | | | | | | |
| Study | | | | | | |
| Groups | | | | | | |
| Group I | 6T, | 43 | 3 | 3 | 48 | — |
| | 4N* | | | | | |
| Group III | 6T, 4N | 58 | 3 | 3 | 46 | 5.5 |
| Validation | 65 | 55.5 | 31 | 22 | 23.5 | |
| Set | | | | | | |
| Study | | | | | | |
| Groups | | | | | | |
| Group I | 34 | 60 | 19 | 9 | 27 | |
| Group II | 19 | 56 | 6 | 10 | 23.5 | 12 |
| Group III | 12 | 48 | 6 | 3 | 20.5 | 4 |
| QRT | 30 | 57 | 14 | 9 | 23 | |
| Group I | 14 | 58 | 7 | 3 | 22 | — |
| Group II | 8 | 58 | 3 | 4 | 15 | 11 |
| Group III | 8 | 50 | 4 | 2 | 9.5 | 3.5 |
| IHC | 35 | 56 | 20 | 13 | 30 | |
| Group I | 20 | 60 | 13 | 6 | 35 | — |
| Group II | 11 | 49 | 4 | 6 | 28 | 16.5 |
| Group III | 4 | 48 | 3 | 1 | 16.5 | 13 |
| Saliva | 37 | 51 | 11 | 14 | | |
| Normal | 12 | 52 | 4 | 6 | — | |
| T1/T2 | 25 | 50 | 7 | 8 | 18 | |

TABLE VII
List of top 10 significant genes in Non-Recurrent/recurrent tongue cancer
Non Recurrent T vs N

| Sl | Affymetrix | Gene | Fold | p | Fold | p |
|---|---|---|---|---|---|---|
| No | ID | Symbol | (NR/Normal) | (NR/Normal) | (R/Normal) | (R/Normal) |
| 1 | 204475_at | MMP1 | 255.50 | 0.00012 | 74.50 | 0.00519 |
| 2 | 213139_at | SNAI2 | 5.81 | 0.00014 | 2.81 | 0.0222 |
| 3 | 202458_at | PRSS23 | 8.77 | 0.000186 | 4.53 | 0.0205 |
| 4 | 205828_at | MMP3 | 35.40 | 0.000288 | 26.15 | 0.0141 |
| 5 | 205680_at | MMP10 | 29.51 | 0.00102 | 23.70 | 0.0151 |
| 6 | 222108_at | AMIGO2 | 5.25 | 0.00224 | 3.27 | 0.024 |
| 7 | 201976_s_at | MYO10 | 3.96 | 0.00396 | 2.21 | 0.0333 |
| 8 | 203936_s_at | MMP9 | 13.60 | 0.00438 | 8.39 | 0.0206 |
| 9 | 225681_at | CTHRC1 | 16.01 | 0.00454 | 9.96 | 0.0378 |
| 10 | 225646_at | CTSC | 7.17 | 0.0058 | 4.66 | 0.0319 |

Recurrent T vs N

| Sl | Affymetrix | Gene | p | Fold | p | Fold |
|---|---|---|---|---|---|---|
| No | ID | Symbol | (R/Normal) | (R/Normal) | (NR/Normal) | (NR/Normal) |
| 1 | 204567_s_at | ABCG1 | 3.83E−05 | 6.71 | 0.00166 | 3.78 |
| 2 | 205479_s_at | PLAU | 0.00409 | 7.66 | 0.00268 | 4.95 |
| 3 | 203562_at | FEZ1 | 0.00837 | 6.14 | 0.036 | 3.20 |
| 4 | 225285_at | BCAT1 | 0.0196 | 6.16 | 0.0265 | 3.98 |
| 5 | 212488_at | COL5A1 | 0.0197 | 7.18 | 0.0117 | 5.88 |
| 6 | 205959_at | MMP13 | 0.0205 | 25.45 | 0.0313 | 10.91 |
| 7 | 202998_s_at | LOXL2 | 0.0206 | 5.31 | 0.0452 | 3.69 |
| 8 | 214297_at | CSPG4 | 0.0249 | 5.44 | 0.0144 | 4.18 |
| 9 | 214329_x_at | TNFSF10 | 0.0303 | 4.09 | 0.0312 | 2.96 |
| 10 | 202688_at | TNFSF10 | 0.036 | 3.96 | 0.0141 | 2.31 |

TABLE VIII
List of significant genes in Recurrent tongue cancer

| | | | Normal | | Tumor | |
|---|---|---|---|---|---|---|
| Sl | Affymetrix | Gene | (NR/R) | | (NR/R) | p- |
| No | ID | Symbol | Fold | p-value | Fold | value |
| 1 | 209116_x_at | HBB | 33.93 | 0.00287 | 11.92 | 0.0462 |
| 2 | 217232_x_at | HBB | 23.81 | 0.00256 | 8.07 | 0.0489 |
| 3 | 209458_x_at | HBA1 /// | 23.19 | 0.0183 | 5.23 | 0.0378 |
| | | HBA2 | | | | |
| 4 | 211745_x_at | HBA1 | 22.72 | 0.0124 | 6.52 | 0.0376 |
| 5 | 211699_x_at | HBA1 /// | 18.70 | 0.0284 | 4.50 | 0.0422 |
| | | HBA2 | | | | |
| 6 | 204018_x_at | HBA1 /// | 16.41 | 0.0232 | 4.61 | 0.0441 |
| | | HBA2 | | | | |
| 7 | 218841_at | ASB8 | 3.72 | 0.048 | 1.66 | 0.00659 |
| 8 | 228222_at | PPP1CB | 3.04 | 0.0382 | 2.07 | 0.0463 |
| 9 | 220952_s_at | PLEKHA5 | −2.29 | 0.0495 | −1.65 | 0.0453 |
| 10 | 208003_s_at | NFAT5 | −2.35 | 0.0397 | −2.44 | 0.014 |
| 11 | 242989_at | — | −2.79 | 0.0108 | −1.53 | 0.0034 |

TABLE IX
Reciever Operating Curve and Regression analysis of the markers

| | | | Asymptotic 95% | | |
|---|---|---|---|---|---|
| | | | Confidence Interval | | |
| ROC Analysis | | | Lower | Upper | |
| Test Result Variable | Area | Std Error | bound | bound | p value |
| COL5A1 | 0.806 | 0.0793 | 0.65 | 0.961 | 0.0001 |
| IGLA | 0.824 | 0.0822 | 0.622 | 0.985 | 0.0001 |
| HBB | 0.975 | 0.0201 | 0.936 | 1.000 | <0.0001 |
| CTSC | 0.746 | 0.0914 | 0.566 | 0.925 | 0.0072 |
| ABCG1 | 0.661 | 0.101 | 0.462 | 0.859 | 0.112 |
| MMP1 | 0.533 | 0.109 | 0.319 | 0.748 | 0.759 |
| EMP1 | 0.464 | 0.11 | 0.249 | 0.679 | 0.745 |
| CCL18 | 0.605 | 0.109 | 0.392 | 0.818 | 0.334 |

| Regression Analysis | | | | |
|---|---|---|---|---|
| Independent variables | Coefficient | Std. Error | t | p |
| (Constant) | −0.02586 | | | |
| COL5A1 | 0.3341 | 0.108 | 3.092 | 0.0046 |
| HBB | 0.6724 | 0.1088 | 6.182 | <0.0001 |

TABLE X
Consolidated List of genes with high differential expression

| Sl | | | Fold | Gene |
|---|---|---|---|---|
| NO | Affymetrix ID | P-value | R/N | Symbol |
| 1 | 204475_at | 0.00519 | 74.50 | MMP1 |
| 2 | 205959_at | 0.0205 | 25.45 | MMP13 |
| 3 | 211964_at | 0.00664 | 11.14 | COL4A2 |
| 4 | 211980_at | 0.0103 | 8.53 | COL4A1 |
| 5 | 221730_at | 0.0179 | 7.79 | COL5A2 |
| 6 | 205479_s_at | 0.00409 | 7.66 | PLAU |
| 7 | 212488_at | 0.0197 | 7.18 | COL5A1 |
| 8 | 204567_s_at | 3.83E−05 | 6.71 | ABCG1 |
| 9 | 225285_at | 0.0196 | 6.16 | BCAT1 |
| 10 | 203562_at | 0.00837 | 6.14 | FEZ1 |
| 11 | 210986_s_at | 0.014 | 5.86 | TPM1 |
| 12 | 209651_at | 0.0105 | 5.46 | TGFB1I1 |
| 13 | 203065_s_at | 0.0194 | 5.31 | CAV1 |
| 14 | 202998_s_at | 0.0206 | 5.31 | LOXL2 |
| 15 | 236565_s_at | 0.0145 | 5.12 | LARP6 |
| 16 | 221261_x_at | 0.0183 | 5.10 | MAGED4 /// |
| | | | | MAGED4B |
| 17 | 208091_s_at | 0.0188 | 4.85 | ECOP |
| 18 | 201185_at | 0.0118 | 4.48 | HTRA1 |
| 19 | 214329_x_at | 0.0303 | 4.09 | TNFSF10 |
| 20 | 221523_s_at | 0.0102 | −4.00 | RRAGD |
| 21 | 223822_at | 0.00826 | −4.18 | SUSD4 |
| 22 | 213895_at | 0.0158 | −4.50 | EMP1 |
| 23 | 218858_at | 0.0199 | −4.59 | DEPDC6 |
| 24 | 231929_at | 0.00177 | −6.58 | IKZF2 |
| 25 | 214063_s_at | 0.0116 | −6.67 | TF |
| 26 | 231145_at | 0.0184 | −7.19 | — |
| 27 | 209498_at | 0.0174 | −7.69 | CEACAM1 |
| 28 | 1559606_at | 0.0192 | −11.51 | GBP6 |
| 29 | 220026_at | 0.00299 | −16.26 | CLCA4 |
| | | | Fold | Gene |
| | Affymetrix ID | P-value | NR/R | Symbol |
| 30 | 209116_x_at | 0.0462 | 11.92 | HBB |
| 31 | 203872_at | 0.0368 | 9.79 | ACTA1 |
| 32 | 204179_at | 0.0466 | 9.45 | MB |
| 33 | 204810_s_at | 0.047 | 7.64 | CKM |
| 34 | 205374_at | 0.0427 | 6.84 | SLN |
| 35 | 209742_s_at | 0.0179 | 6.82 | MYL2 |
| 36 | 211745_x_at | 0.0376 | 6.52 | HBA1 |
| 37 | 209888_s_at | 0.0497 | 6.38 | MYL1 |
| 38 | 211959_at | 0.00223 | 5.81 | IGFBP5 |
| 39 | 224646_x_at | 0.0311 | 5.66 | H19 |
| 40 | 209904_at | 0.0386 | 5.12 | TNNC1 |
| 41 | 219772_s_at | 0.0458 | 4.95 | SMPX |
| 42 | 202037_s_at | 0.0491 | 4.64 | SFRP1 |
| 43 | 209283_at | 0.00949 | 3.76 | CRYAB |
| 44 | 209355_s_at | 0.014 | 3.68 | PPAP2B |
| 45 | 212654_at | 0.043 | 3.51 | TPM2 |
| 46 | 202036_s_at | 0.0409 | 3.43 | SFRP1 |
| 47 | 243720_at | 0.0395 | −1.91 | CMIP |
| 48 | 228310_at | 0.0249 | −1.92 | ENAH |
| 49 | 208614_s_at | 0.000933 | −2.04 | FLNB |
| 50 | 208003_s_at | 0.014 | −2.44 | NFAT5 |
| 51 | 204475_at | 0.00012 | 255.50 | MMP1 |
| 52 | 211430_s_at | 0.00483 | 41.16 | IGH@ /// |
| | | | | IGHG1 /// |
| | | | | IGHG2 /// |
| | | | | IGHG3 /// |
| | | | | IGHM /// |
| | | | | IGHV4-31 |
| 53 | 209138_x_at | 0.00186 | 36.17 | IGL@ |
| 54 | 205828_at | 0.000288 | 35.40 | MMP3 |
| 55 | 205680_at | 0.00102 | 29.51 | MMP10 |
| 56 | 201645_at | 0.000184 | 28.77 | TNC |
| 57 | 211756_at | 0.000497 | 28.52 | PPIA |
| 58 | 215121_x_at | 0.00254 | 27.28 | PABPC1 |
| 59 | 209395_at | 0.00282 | 24.94 | CHI3L1 |
| 60 | 215379_x_at | 0.00111 | 24.04 | LOX |
| 61 | 209924_at | 0.000224 | 21.57 | CCL18 |
| 62 | 202267_at | 0.00441 | 16.25 | LAMC2 |
| 63 | 225681_at | 0.00454 | 16.01 | CTHRC1 |
| 64 | 218468_s_at | 0.000843 | 14.26 | GREM1 |
| 65 | 32128_at | 0.000984 | 13.70 | TREX1 |
| 66 | 203936_s_at | 0.00438 | 13.60 | MMP9 |
| 67 | 210355_at | 0.000551 | 13.36 | PTHLH |
| 68 | 221671_x_at | 0.00132 | 13.29 | CLEC7A |
| 69 | 221651_x_at | 0.00283 | 13.19 | ARHGEF10L |
| 70 | 204533_at | 0.00187 | 11.34 | CXCL10 |
| 71 | 215446_s_at | 0.000434 | 10.80 | SEC16A |
| 72 | 225647_s_at | 7.29E−05 | 9.66 | UHRF1 |
| 73 | 203915_at | 0.00128 | 9.54 | CXCL9 |
| 74 | 20245 8_at | 0.000186 | 8.77 | PRSS23 |
| 75 | 206513_at | 0.000704 | 8.65 | AIM2 |
| 76 | 206026_s_at | 0.000441 | 7.44 | FSCN1 |
| 77 | 205159_at | 0.00094 | 6.79 | CSF2RB |
| 78 | 201422_at | 0.000631 | 6.50 | IFI30 |
| 79 | 212364_at | 7.84E−05 | 6.38 | MYO1B |
| 80 | 201579_at | 0.000503 | 6.37 | FAT |
| 81 | 213139_at | 0.00014 | 5.81 | SP3 |
| 82 | 213139_at | 0.00014 | 5.81 | SNAI2 |
| 83 | 226368_at | 0.000587 | 5.74 | CHST11 |
| 84 | 221898_at | 0.0022 | 5.74 | CYLD |
| 85 | 209360_s_at | 0.000443 | 5.55 | RUNX1 |
| 86 | 203417_at | 0.00102 | 5.44 | MFAP2 |
| 87 | 229400_at | 0.0015 | 5.44 | IFIT3 |
| 88 | 222108_at | 0.00224 | 5.25 | GPR172A |
| 89 | 222108_at | 0.00224 | 5.25 | AMIGO2 |
| 90 | 203423_at | 0.00155 | 5.25 | RBP1 |
| 91 | 212588_at | 0.00348 | 5.19 | RRAS2 |
| 92 | 221059_s_at | 0.00174 | 5.15 | TXNDC5 |
| 93 | 204972_at | 0.00295 | 5.15 | OAS2 |
| 94 | 218400_at | 0.003 | 5.05 | SNX10 |
| 95 | 202953_at | 0.000743 | 5.01 | C1QB |
| 96 | 212365_at | 0.00127 | 4.77 | GART |
| 97 | 204222_s_at | 0.000446 | 4.65 | GLIPR1 |
| 98 | 201487_at | 0.00284 | 4.52 | CTSC |
| 99 | 202558_s_at | 0.000662 | 4.50 | STCH |
| 100 | 201564_s_at | 0.00094 | 4.45 | FSCN1 |
| 101 | 206584_at | 0.000407 | 4.44 | LY96 |
| 102 | 201853_s_at | 0.00253 | 4.35 | CDC25B |
| 103 | 203083_at | 0.00134 | 4.34 | THBS2 |
| 104 | 201818_at | 0.000494 | 4.34 | LPCAT1 |
| 105 | 204362_at | 0.000204 | 4.29 | SKAP2 |
| 106 | 201417_at | 0.00397 | 4.20 | SOX4 |
| 107 | 226372_at | 0.000739 | 4.18 | ERGIC2 |
| 108 | 200644_at | 0.00221 | 4.10 | MARCKSL1 |
| 109 | 219298_at | 0.00438 | −5.75 | DERL1 |

1. A novel molecular signature comprising of gene expression profile of a combination of two or more genes from the set ABCA1, ABCE1, ABCG1, ABHD12, ACLY, ACOT9, ACTA1, ACTL6A, ACTN1, ACTR1B, ACTR2, ADAM17, ADAM9, ADAR, AHR, AIM2, AKR1B1, AKTIP, ALG3, ALKBH7, AMD1, AMIGO2, AMMECR1L, ANKH, ANKRD11, ANKRD50, ANP32E, ANXA1, AP3S2, APLP2, APOBEC3A, APOE, APOL6, APP, ARF3, ARFIP1, ARFIP2, ARHGAP5, ARHGEF1, ARHGEF10L, ARL1, ARMC10, ARPC1B, ARSI, ARVCF, ASB8, ASPH, ATP2B4, ATP2C1, ATP6V0E1, ATP6V1C1, ATP8B1, ATR, AURKA, BAG1, BANF1, BASP1, BAZ1A, BAZ2B, BBC3, BCAM, BCAT1, BCL6, BCLAF1, BID, BMS1, BRD1, BSCL2, BUB3, BXDC5, CA13, CADM1, CALU, CAMSAP1L1, CAND1, CASP1, CASP7, CAV1, CBLB, CBX3, CCDC128, CCDC44, CCL18, CCNE1, CCNF, CDC25B, CDC27, CDC34, CDK6, CDKN2A, CEACAM1, CENTB2, CENTD1, CENTD2, CFB, CHFR, CHI3L1, CHMP6, CHST11, CHSY1, CHUK, CHURC1, CIDEB, CIRH1A, CKLF, CKM, CLCA4, CLCC1, CLCN7, CLDND1, CLEC7A, CLPP, CLPX, CMIP, CMTM4, COBL, COL4A1, COL4A2, COL5A1, COL5A2, COPE, COQ9, COTL1, COX15, CRYAB, CSDA, CSF2RB, CSGlcA-T, CSNK1A1, CSNK2A2, CSPG4, CTHRC1, CTNND1, CTSB, CTSC, CXCL10, CXCL16, CXCL9, CXXC5, CYBASC3, CYLD, CYP51A1, DCBLD1, DCLRE1C, DCXR, DDOST, DDX21, DDX, DDX54, DEPDC6, DERL1, DFNA5, DGKQ, DHRS7B, DLG7, DLGAP4, DPY19L3, DRAM, DTX3L, E2F3, EAF2, ECHDC2, ECOP, ECSIT, EFHD2, EGFL6, EIF2S3, EIF3G, EIF4E, EIF5, EMILIN2, EMP1, ENAH, ENSA, ENTPD6, EPSTI1, ERCC1, ERGIC2, ERGIC3, ERMP1, ETV6, EZH2, FAM101B, FAM116A, FAM122B, FAM122B, TMEM57, FAM125A, FAM135A, FAM33A, FAM38A, FAM3A, FAM80B, FAM89B, FAM91A1, FAT, FBLIM1, FBRS, FBXW5, FEZ1, FJX1, FKBP15, FKBP2, FKBP9, FLJ21438, FLJ35220, FLNB, FLRT2, FNDC3B, FOLR1, FOLR2, FOXJ2, FRMD4B, FRMD6, FSCN1, FST, FTSJ1, FUS, FUT7, GALNAC4S-6ST, GART, GASS, SNORD79, GBP6, GJA1, GLIPR, GLTP, GLTSCR2, GLUD1, GMD, GNAl2, GNB5, GNPTG, GOLGA8A, GPD1L, GPD2, GPR108, GPR137B, GPR172A, GPR176, GPR5, GPSM1, GREM1, GSDMDC1, GSK3A, GSPT1, GTF2A1, GZMB, H19, H2AFX, HAB1, HAX1, HBA1, HBA1, HBA2, HBA2, HBB, RAP1B, HEATR1, HEATR6, HERC5, HEXB, HIBCH, HIF1A, HINT2, HIPK1, HIST1H1C, HMCN2, HNRNPA2B1, HNRNPC, HNRPA3, HOPX, HRASLS, HRB, HSP90B1, HSPB8, HSPH1, HTRA1, ICT1, IDH3G, IF116, IFI30, IF16, IFIT3, IFNGR1, IGF2BP2, IGFBP5, IGH@, IGHG1, IGHG2, IGHG3, IGHM, IGHV4-31, IGK@, IGKC, IGKV1-5, IGKV2-24, IGLJ3, IGLV2-14, IGL@, IGLV325, IKZF2, IL10RB, IL1R1, IL8, IMP4, IMPDH1, IRAK1, IRF9, ITGA6, JAG2, JOSD3, KCNS3, KDELR2, KIAA0241, KIAA0494, KIAA0562, KIAA0746, KIAA0922, KIAA1468, KIAA1546, KIAA1598, KIDINS220, KIF21A, KIF3B, KIRREL, KLHL22, KPNA1, KPNA2, KPNA3, KRBA1, LAMC2, LAPTM5, LARP6, LASP1, LBR, LHFPL2, LIMA1, LMAN2, LMAN2L, LMNA, LMOD2, THAP4, LOC729604, LOX, LOXL2, LPCAT1, LRIG3, LRRC58, LRRC8D, LSG1, LTBP3, LY96, LYAR, MAGED1, MAGED4, MAGED4B, MAMDC2, MAML2, MAN2A1, MAN2B1, MAOB, MAP1LC3B, MAP4K5, MAPRE3, MARCKS, MARCKSL1, MARVELD1, MATR3, MAX, MAZ, MB, MEF2A, MEX3C, MFAP2, MFHAS1, MFSD5, MGC3196, MIB1, MIB2, MIER1, MINA, MIS12, MLLT11, MLSTD2, MLXIPL, MMP1, MMP10, MMP12, MMP13, MMP3, MMP9, MOBKL1A, MRPL48, MRPS18B, MTCP1, MTHFD2, MVP, MYBL2, MYL1, MYL2, MY010, MYO1B, N4BP1, NADK, NARS2, NAT10, NAV2, NBN, NCOA1, NDE1, NDRG2, NEK6, NETO2, NFAT5, NFATC2IP, NINJ1, NIPSNAP3A, NOL5A, NOTCH2, NPAT, NPEPL1, NPL, NQO1, NSFL1C, NUAK1, NUBP2, NUDCD1, NUDT22, NXT1, OAS2, OAS3, ODZ2, OFD1, OGT, OSTM1, OXA1L, PABPC1, PAFAH1B2, PAPD4, PARN, PCDHGA1, PCDHGA10, PCDHGA11, PCDHGA12, PCDHGA2, PCDHGA3, PCDHGA4, PCDHGA5, PCDHGA6, PCDHGA7, PCDHGA8, PCDHGA9, PCDHGB1, PCDHGB2, PCDHGB, PCDHGB4, PCDHGB5, PCDHGB6, PCDHGB7, PCDHGC3, PCDHGC4, PCDHGC5, PCTK2, PDCD2L, PDGFC, PDPN, PEMT, PET112L, PFN2, PGGT1B, PGS1, PHLDB1, PIK3CD, PIK3R4, PIP5K3, PKN2, PKP4, PLAU, PLEKHA5, PLEKHG5, PLK2, PLOD3, PLXDC2, PLXNA1, PNMA1, PNN, PNPLA2, PNPLA4, PNRC1, POLD4, POLR1D, POLR2G, POLR2J, POPS, PPAP2B, PPFIA1, PPIA, PPP1CA, PPP1CB, PPP2R1B, PRDM1, PRNP, PROCR, PRPF38A, PRSS23, PSMA3, PSMA4, PSMD8, PSME2, PSME3, PSMF1, PTCD1, PTHLH, PTPN2, PTPRE, PTPRK, PTPRZ1, PUS7, PXDN, PXMP4, RAB23, RAB31, RAB32, RABGAP1L, RABL5, RAD51C, RAPGEFL1, RAPH1, RASGEF1A, RBBP4, RBM17, RBM22, RBM25, RBMS1, RBMX, RBP1, RBP7, RC3H2, RDH11, REEP3, RER1, RFFL, RGS3, RGS4, RHBDF2, RHOQ, RHOU, RILP, RIN2, RIPK2, RNASEH2A, RNF12, RNF138, RNF145, RNF167, RP6, 213H19.1, RPN2, RPP25, RRAGD, RRAS2, RRP15, RSPRY1, RTP4, RUNX1, RYBP, SAC3D1, SART3, SCHIP1, SCRN1, SDAD1, SDF2, SDF4, SEC16A, SEC23B, SEC24A, SEC63, SEP15, SERPINB1, SERPINH1, SFPQ, SFRP1, SFRS12, SFRS2, SFRS7, SFXN3, SFXNS, SGMS2, SHARPIN, SHC1, SIRPA, SIXS, SKAP2, SLC16A3, SLC25A13, SLC26A2, SLC2A3P1, SLC30A7, SLC35B1, SLC39A14, SLC39A6, SLC44A2, SLC6A2, SLC7A6, SLN, SMPD1, SMPX, SMYD2, SNAI2, SNAPC1, SNX10, SOD3, SORT1, SOX15, SOX4, SP110, SP3, SPIRE1, SRP72, SRPK1, SRPK2, SRPR, SSSCA1, ST3GAL5, STAT1, STAT2, STAT3, STCH, STK38, STMN1, STRBP, SUDS3, SUSD4, SYNGR1, SYNJ2, TAF10, TAP2, TARBP2, TARS, TCERG1, TDG, TES, TEX10, TFTFCP2L1, TFE3, TGFB1I1, TGIF1, THAP11, THBS2, THEM2, THSD1, TIGDS, TIMM17B, TLR2, TMCC1, TMED10, TMED8, TMEM123, TMEM161B, TMEM170, TMEM184B, TMEM189, TMEM29, TMEM39B, TMEPAI, TMTC3, TNC, TNFAIP6, TNFRSF1A, TNFSF10, TNNC1, TNNT1, TOM1L1, TOPBP1, TP53I11, TP53INP1, TPBG, TPM1, TPM2, TPST1, TRABD, TRAM2, TRAPPC1, TREX1, TRIM22, TRIM38, TRIM47, TRIO, TRMT6, TUBA1C, TWF2, TXNDC1, TXNDC12, TXNDCS, UBE2L6, UBL3, UCK1, UCKL1, UFM1, UHRF1, URM1, USE1, USP1, USP34, USP53, UTP11L, VARS2, VPS24, VPS54, VRK3, WAPAL, WBP1, WDR45, WDR54, WDR68, WDR81, WEE1, WHSC1, XPOT, YBX1, YES1, YKT6, YRDC, ZCCHC17, ZFP64, ZMYM2, ZNF217, ZNF408, ZNF574, ZNFX1, ZSWIM6 or expression of proteins encoded by these genes in carcinoma tissues or tissue adjacent to the carcinoma tissue that is useful for personalizing cancer treatment.

2. The molecular signature as claimed in claim 1 wherein the said molecular signature is used for predicting recurrence of cancer after surgery or treatment with anti-cancer agents or anti cancer therapy.

3. The molecular signature as claimed in claim 1 wherein the molecular signature is used for predicting sensitivity or resistance to anti-cancer agents or anti-cancer therapy.

4. The molecular signature as claimed in claim 1 wherein the molecular signature is used for predicting cancer metastasis at the time of cancer diagnosis to enable appropriate treatment, surgical or non-surgical.

5. The molecular signature as claimed in claim 1 wherein the cancer type includes but is not limited to oral cancer, other head and neck cancers, pancreatic cancer, breast cancer, glioma, melanoma, neuroblastoma, cancers of the gastro-intestinal tract, lung cancer, endometrial cancer, prostate cancer, renal cancer, bone cancer, hepatocellular carcinoma, endocrine cancer, ovarian cancer, and other solid cancers.

6. The molecular signature as claimed in claim 1 wherein molecular signature is derived from cancer tissue samples or tissue adjacent to the cancer tissue samples or saliva, which are either collected in RNA stabilizing solutions, or are frozen samples, fresh samples or formalin fixed paraffin embedded samples.

7. The molecular signature as claimed in claim 1 wherein the molecular signature is identified by techniques including, but not limited to, DNA microarray, quantitative real-time PCR, immunohistochemistry, proteomic analysis, or enzyme linked immunosorbent assay.
